(19) United States
(12) Patent Application Publication
(10) Pub. No.: US 2024/0173000 A1
(43) Pub. Date: May 30, 2024

(54) BLOOD-PRESSURE-RELATED INFORMATION DISPLAY DEVICE

(71) Applicant: OMRON HEALTHCARE CO., LTD., Muko-shi (JP)

(72) Inventors: Yukiya SAWANOI, Kyoto (JP); Shingo Yamashita, Kyoto (JP); Terumasa Naito, Kyoto (JP)

(73) Assignee: OMRON HEALTHCARE CO., LTD., Muko-shi (JP)

(21) Appl. No.: 18/435,596

(22) Filed: Feb. 7, 2024

Related U.S. Application Data
(63) Continuation of application No. PCT/JP2021/039625, filed on Oct. 27, 2021.

(51) Int. Cl. A61B 5/00; A61B 5/022

(52) U.S. Cl. CPC A61B 5/742; A61B 5/022; A61B 5/7221; A61B 2562/0247

(57) ABSTRACT

A blood-pressure-related information display device determines whether a measured value is classified into any of a first range preset in a specification, a second range preset adjacent to a side lower than the first range, and a third range preset adjacent to a side lower than the second range. On a display screen, the measured value is displayed when the measured value is classified into the first range, deviation information indicating that the measured value is classified into the second range or the measured value in addition to the deviation information is displayed when the measured value is classified into the second range, and an indication of measurement error is displayed when the measured value is classified into the third range.

### CROSS-REFERENCE TO RELATED APPLICATIONS

This is a continuation application of International Application No. PCT/JP2021/039625, with an International filing date of Oct. 27, 2021, the entire content of which is hereby incorporated by reference.

### TECHNICAL FIELD

The present invention relates to a blood-pressure-related information display device, and more particularly to a device that displays information related to blood pressure such as a blood pressure value and a pulse rate on a display screen.

### BACKGROUND ART

In a general electronic sphygmomanometer, for example, as described on page 47 (specification) of Non-patent Document 1 (“Upper Arm Type Sphygmomanometer HCR-7501T Instruction Manual”, OMRON HEALTHCARE Co., Ltd.), a maximum blood pressure measurement range is 60 to 260 mmHg, a minimum blood pressure measurement range is 40 to 215 mmHg, and a pulse rate measurement range is 40 to 180 beats/minute. These measurement ranges are preset as a range in which a manufacturer evaluates a sphygmomanometer and measurement accuracy of the sphygmomanometer can be guaranteed (more specifically, a range evaluated and verified using a measured value by an auscultation method by a doctor, which is an evaluation method of an electronic sphygmomanometer, as a reference value).

In the sphygmomanometer, in a case where a maximum blood pressure, a minimum blood pressure, and a pulse rate acquired by measurement are in a corresponding measurement range, for example, as illustrated in FIG. 8A, the acquired maximum blood pressure, minimum blood pressure, and pulse rate are displayed on a display screen. In the example of FIG. 8A, an acquired maximum blood pressure (systolic blood pressure) SYS is 124 mmHg, which falls within a maximum blood pressure measurement range of 60 to 260 mmHg. An acquired minimum blood pressure (diastolic blood pressure) DIA is 74 mmHg, which falls within a minimum blood pressure measurement range of 40 to 215 mmHg. Further, an acquired pulse rate PLS is 61 beats/minute, which falls within a pulse rate measurement range of 40 to 180 beats/minute. In response to this, the acquired maximum blood pressure, minimum blood pressure, and pulse rate are displayed on a display screen.

On the other hand, in the sphygmomanometer, in a case where any one of the maximum blood pressure, the minimum blood pressure, and the pulse rate acquired by measurement deviates from a corresponding measurement range, for example, as illustrated in FIG. 8D, an indication of measurement error (in this example, a character string of “Err”) is displayed on a display screen. For example, even in a case where the acquired maximum blood pressure SYS is 75 mmHg and falls within the maximum blood pressure measurement range of 60 to 260 mmHg, and the acquired minimum blood pressure DIA is 41 mmHg and falls within the minimum blood pressure measurement range of 40 to 215 mmHg, when the acquired pulse rate PLS is 39 beats/minute and deviates from the pulse rate measurement range of 40 to 180 beats/minute, not only the pulse rate but also the maximum blood pressure and the minimum blood pressure are not displayed on the display screen, and the indication of measurement error as illustrated in FIG. 8D is displayed on the display screen. Note that the character strings “SYS”, “DIA”, and “PLS” are fixed in the display screen. In the example of FIG. 8D, the character string “Err” is displayed to the right of the character string “SYS”, but such a position of the character string “Err” has no meaning.

### SUMMARY OF THE INVENTION

In recent years, an electronic sphygmomanometer capable of measuring blood pressure at night has been developed. A blood pressure at night is considered to have a higher correlation with heart disease risk than a blood pressure in the daytime, and is also an important index for knowing an effect of antihypertensive therapy. It is known that a blood pressure and a pulse rate at night are generally lower than a blood pressure and a pulse rate in the daytime in a healthy person, and it is also known that a blood pressure may be even lower in a hypertensive patient or the like who is receiving antihypertensive therapy together with an effect of medicine. This fact is also confirmed by a measurement result by the present inventor (FIG. 19 illustrates measured values of the maximum blood pressure SYS, the minimum blood pressure DIA, and the pulse rate PLS during the night for a certain subject. Further, FIG. 20 illustrates measured values of the maximum blood pressure SYS, the minimum blood pressure DIA, and the pulse rate PLS during the night for another subject). In these cases, in the general electronic sphygmomanometer, even when measurement operation itself is normally performed, an acquired measured value (maximum blood pressure, minimum blood pressure, and/or pulse rate) deviates from a preset measurement range. For this reason, there is a problem that an indication of measurement error is displayed and any information on a blood pressure of a subject is not displayed.

In view of the above, an object of the present invention is to provide a blood-pressure-related information display device capable of displaying information on a blood pressure of a subject even in a case where an acquired measured value deviates from a measurement range (this is referred to as a “first range”) set in advance in a specification.

In order to achieve the object, a blood-pressure-related information display device of the present disclosure is a blood-pressure-related information display device that displays information related to a blood pressure on a display screen, the blood-pressure-related information display device comprising:
a measured value acquisition unit that acquires a measured value including a systolic blood pressure, a diastolic blood pressure, and/or a pulse rate of a subject;
a determination unit that determines which of three ranges the measured value is classified into, the three ranges being a first range preset in a specification, a second range corresponding to a measured value that can be exhibited by a human, preset adjacent to a side lower than the first range, and a third range corresponding to an abnormal measured value that cannot be exhibited by a human, preset adjacent to a side lower than the second range; and
a display processing unit that performs processing of displaying information on a display screen, wherein
the display processing unit:
displays the measured value on the display screen when the measured value is classified into the first range;
displays deviation information indicating that the measured value is classified into the second range or the measured value in addition to the deviation information on the display screen when the measured value is classified into the second range; and
displays an indication of measurement error on the display screen with displaying neither the deviation information nor the measured value when the measured value is classified into the third range.

A “measured value including a systolic blood pressure, a diastolic blood pressure, and/or a pulse rate” includes a case of a measured value of any one measurement item of a systolic blood pressure, a diastolic blood pressure, and a pulse rate and a case of a measured value of a plurality of measurement items.

To “acquire a measured value” includes, for example, a case of acquiring a measured value by performing measurement in the present device and a case of acquiring a measured value that is measured outside the present device and input via, for example, a communication line.

A “first range set by a specification” may be substantially set by an instruction manual of a device or the like without limitation to a specification sheet.

A “human” refers to a general person, and may be a plurality of persons. A “human” may include a “subject”.

A “second range corresponding to a measured value that may be exhibited by a human, preset adjacent to a side lower than the first range” is preset based on an empirical fact so as to include distribution of measured values that may be exhibited, for example, when a human is sleeping at night, when a human drinks alcohol and a blood vessel expands, when a medicine (for example, a hypotensive agent) taken by a human is too effective, and the like.

Further, a “third range” is preset based on an empirical fact as a range of abnormal measured values that cannot be exhibited by a human.

“Deviation information” may be displayed, for example, as a character, a mark, or the like indicating deviation, or may be displayed as a specific mode of the measured value.

### BRIEF DESCRIPTION OF THE DRAWINGS

FIG. 1 is a diagram illustrating a block configuration of a sphygmomanometer as an embodiment to which a blood-pressure-related information display device of the present invention is applied.

FIG. 2 is a diagram illustrating an operation flow of the sphygmomanometer.

FIG. 3 is a diagram illustrating distribution of measured values of a pulse rate PLS that can be exhibited by a human on a lower side and a higher side than a measurement range (first range) set in advance in a specification.

FIG. 4A is a diagram illustrating a classification criterion table defining first to fifth ranges stored in a memory of the sphygmomanometer. FIG. 4B is a diagram illustrating a flag value table that defines flag values and is stored in the memory of the sphygmomanometer.

FIG. 5A is a diagram illustrating contents displayed on a display screen when all measured values of a maximum blood pressure SYS, a minimum blood pressure DIA, and a pulse rate PLS of a subject measured by the sphygmomanometer are classified into the first range. FIGS. 5B and 5C are diagrams illustrating contents displayed on the display screen of the sphygmomanometer when measured values of the maximum blood pressure SYS and the minimum blood pressure DIA of a subject measured by the sphygmomanometer are classified into the first range and a measured value of the pulse rate PLS of the subject is classified into the second range. FIGS. 5D and 5E are diagrams illustrating contents displayed on the display screen of the sphygmomanometer when measured values of the maximum blood pressure SYS and the minimum blood pressure DIA of a subject measured by the sphygmomanometer are classified into the first range and a measured value of the pulse rate PLS of the subject is classified into the fourth range.

FIGS. 6A and 6B are diagrams illustrating contents displayed on the display screen of the sphygmomanometer when measured values of the maximum blood pressure SYS and the minimum blood pressure DIA of a subject measured by the sphygmomanometer are classified into the first range and a measured value of the pulse rate PLS of the subject is classified into the second range. FIGS. 6C and 6D are diagrams illustrating contents displayed on the display screen of the sphygmomanometer when measured values of the maximum blood pressure SYS and the minimum blood pressure DIA of a subject measured by the sphygmomanometer are classified into the first range and a measured value of the pulse rate PLS of the subject is classified into the fourth range.

FIGS. 7A and 7B are diagrams illustrating contents displayed on the display screen of the sphygmomanometer when a measured value of the minimum blood pressure DIA of a subject measured by the sphygmomanometer is classified into the first range and measured values of the maximum blood pressure SYS and the pulse rate PLS of the subject are classified into the fourth range. FIGS. 7C and 7D are diagrams illustrating contents displayed on the display screen of the sphygmomanometer when all measured values of the maximum blood pressure SYS, the minimum blood pressure DIA, and the pulse rate PLS of a subject measured by the sphygmomanometer are classified into the second range.

FIG. 8A is a diagram illustrating contents displayed on the display screen of the sphygmomanometer when all measured values (the maximum blood pressure SYS, the minimum blood pressure DIA, and the pulse rate PLS) of a subject measured by the sphygmomanometer are classified into the first range. FIGS. 8B and 8C are diagrams illustrating contents displayed on the display screen of the sphygmomanometer when measured values of the maximum blood pressure SYS and the minimum blood pressure DIA of a subject measured by the sphygmomanometer are classified into the first range and a measured value of the pulse rate PLS of the subject is classified into the second range. FIG. 8D is a diagram illustrating contents displayed on the display screen of the sphygmomanometer when any of measured values of the maximum blood pressure SYS, the minimum blood pressure DIA, and the pulse rate PLS of a subject measured by the sphygmomanometer is classified into the third or fifth range.

FIG. 9 is a diagram illustrating a schematic configuration of a medical information management system as another embodiment to which the blood-pressure-related information display device of the present invention is applied.

FIG. 10 is a diagram illustrating a block configuration of a medical institution terminal included in the system.

FIG. 11 is a diagram illustrating an operation flow of a sphygmomanometer included in the system.

FIG. 12 is a diagram illustrating an operation flow of the medical institution terminal cooperating with the operation flow of FIG. 11 of the sphygmomanometer.

FIGS. 13A to 13E are diagrams each illustrating a data set transmitted from the sphygmomanometer included in the system to the medical institution terminal.

FIG. 14, portion (A) is a diagram illustrating contents displayed on a display screen of the sphygmomanometer when all measured values of the maximum blood pressure SYS, the minimum blood pressure DIA, and the pulse rate PLS of a subject measured by the sphygmomanometer are classified into the first range. FIG. 14, portion (B) is a diagram illustrating contents displayed on a display screen of the medical institution terminal when all measured values of the maximum blood pressure SYS, the minimum blood pressure DIA, and the pulse rate PLS of a subject measured by the sphygmomanometer are classified into the first range as in FIG. 14, portion (A).

FIG. 15, portion (B) and FIG. 15, portion (C) are diagrams illustrating contents displayed on the display screen of the medical institution terminal when measured values of the maximum blood pressure SYS and the minimum blood pressure DIA of a subject are classified into the first range and a measured value of the pulse rate PLS of the subject is classified into the second range in the sphygmomanometer. FIG. 15, portion (D) and FIG. 15, portion (E) are diagrams illustrating contents displayed on the display screen of the medical institution terminal when a measured value of the minimum blood pressure DIA of a subject measured by the sphygmomanometer is classified into the first range and measured values of the maximum blood pressure SYS and the pulse rate PLS of the subject are classified into the fourth range. FIG. 15, portion (F) and FIG. 15, portion (G) are diagrams illustrating contents displayed on the display screen of the medical institution terminal when measured values of the maximum blood pressure SYS and the minimum blood pressure DIA of a subject measured by the sphygmomanometer are classified into the first range and a measured value of the pulse rate PLS of the subject is classified into the fourth range. FIG. 15, portion (A) is a diagram illustrating contents displayed on the display screen of the sphygmomanometer when contents of FIG. 15, portion (B) to FIG. 15, portion (G) is displayed on the display screen of the medical institution terminal.

FIG. 16, portion (A) is a diagram illustrating contents displayed on the display screen of the sphygmomanometer when any of measured values of the maximum blood pressure SYS, the minimum blood pressure DIA, and the pulse rate PLS of a subject measured by the sphygmomanometer is classified into the third or fifth range. FIG. 16, portion (B) is a diagram illustrating contents displayed on the display screen of the medical institution terminal when any of measured values of the maximum blood pressure SYS, the minimum blood pressure DIA, and the pulse rate PLS of the subject measured by the sphygmomanometer is classified into the third or fifth range as in FIG. 16, portion (A).

FIG. 17 is a diagram illustrating another operation flow of a sphygmomanometer included in the system.

FIG. 18 is a diagram illustrating an operation flow of the medical institution terminal cooperating with the operation flow of FIG. 17 of the sphygmomanometer.

FIG. 19 is a diagram illustrating measured values of the maximum blood pressure SYS, the minimum blood pressure DIA, and the pulse rate PLS during the night for a certain subject.

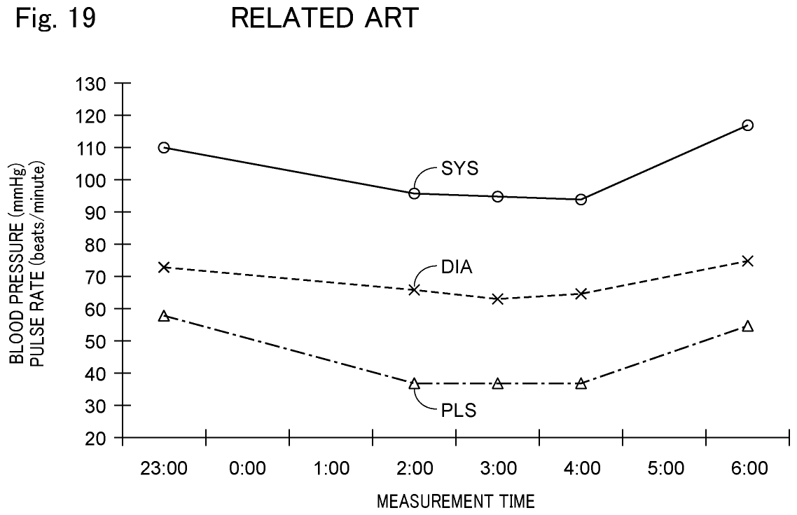

FIG. 20 is a diagram illustrating measured values of the maximum blood pressure SYS, the minimum blood pressure DIA, and the pulse rate PLS during the night for another subject.

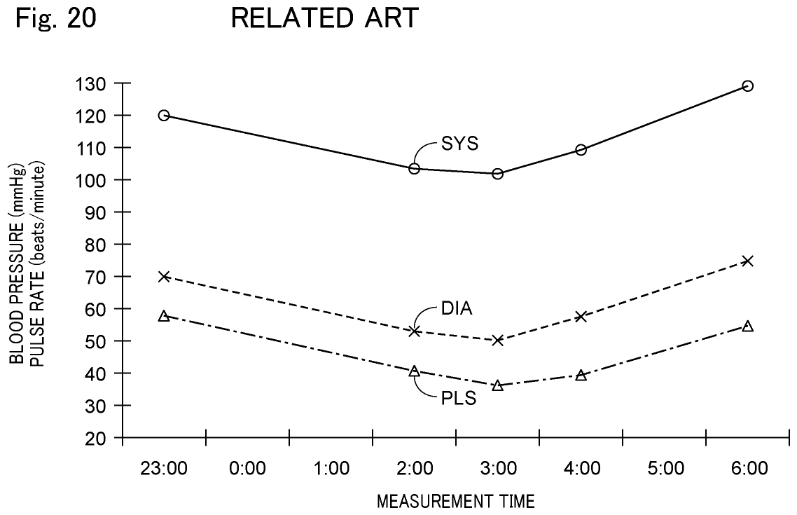

### DETAILED DESCRIPTION

Hereinafter, embodiments of the present invention will be described in detail with reference to the drawings.

### First Embodiment

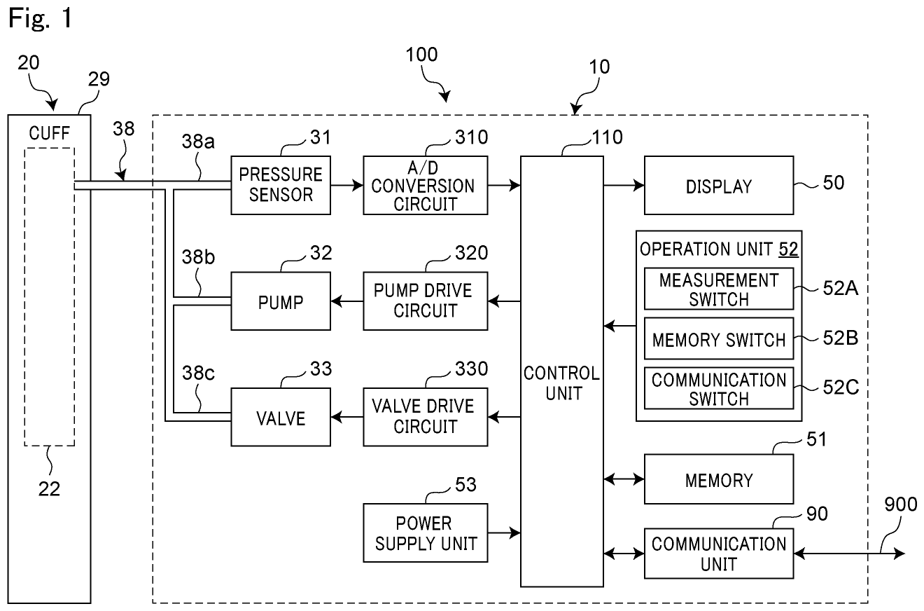

FIG. 1 illustrates a block configuration of a sphygmomanometer 100 as an embodiment to which a blood-pressure-related information display device of the present invention is applied.

The sphygmomanometer 100 roughly includes a cuff 20 for measuring a blood pressure attached around a measurement target site such as an upper arm or a wrist of a subject, and a main body 10 connected to the cuff 20 via an air pipe 38 in a fluid communicable manner. The measurement target site is typically an upper arm, but is not limited to an upper arm, and may be an upper limb other than an upper arm such as a wrist or a lower limb such as an ankle.

The cuff 20 is a general cuff, and includes a bag-shaped band body 29 extending in an elongated shape and a fluid bag 22 contained in the band body 29.

The main body 10 includes a control unit 110, a display 50, an operation unit 52, a memory 51 as a storage unit, a power supply unit 53, a pressure sensor 31, an A/D conversion circuit 310, a pump 32, a pump drive circuit 320, a valve 33, and a valve drive circuit 330. In this example, an air pipe 38a connected to the pressure sensor 31, an air pipe 38b connected to the pump 32, and an air pipe 38c connected to the valve 33 merge to form one air pipe 38 connected to the fluid bag 22 in the cuff 20 in a fluid communicable manner. Hereinafter, the air pipes 38a, 38b, and 38c are collectively referred to as the air pipe 38.

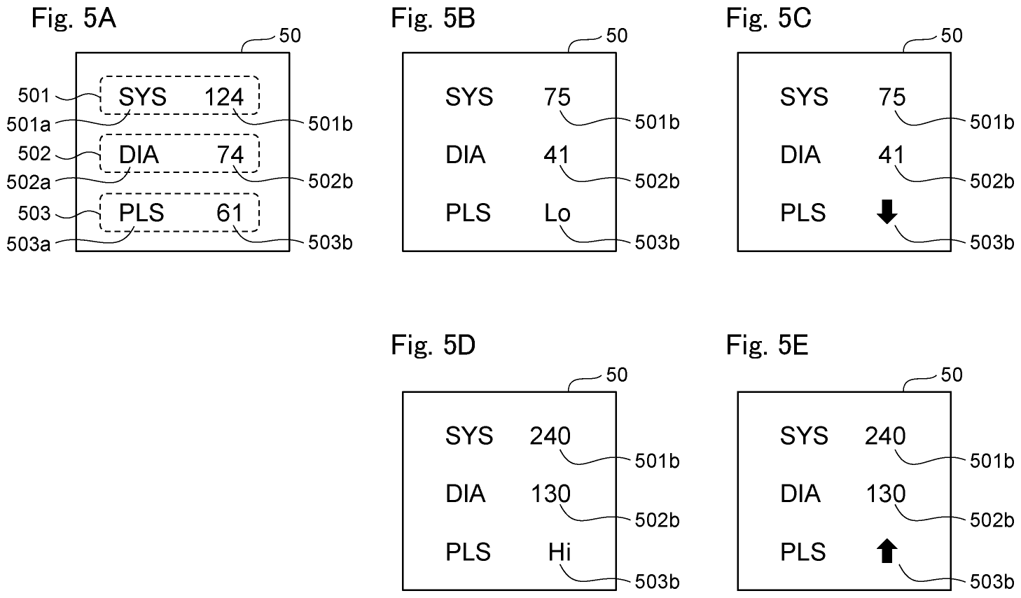

In this example, the display 50 includes a liquid crystal display (LCD), and displays predetermined information in accordance with a control signal from the control unit 110. In this example, as illustrated in FIG. 5A, a display screen of the display 50 is provided with, in order from the top, a maximum blood pressure display region 501 for displaying a maximum blood pressure SYS (systolic blood pressure, units; mmHg), a minimum blood pressure display region 502 for displaying a minimum blood pressure DIA (diastolic blood pressure, units; mmHg), and a pulse rate display region 503 for displaying a pulse rate PLS (units; beat/minute). Character strings “SYS”, “DIA”, and “PLS” are fixedly displayed in a left side portion 501a of the maximum blood pressure display region 501, a left side portion 502a of the minimum blood pressure display region 502, and a left side portion 503a of the pulse rate display region 503, respectively. In each of a right side portion 501b of the maximum blood pressure display region 501, a right side portion 502b of the minimum blood pressure display region 502, and a right side portion 503b of the pulse rate display region 503, an acquired measured value is normally displayed each time a blood pressure is measured by the sphygmomanometer 100. Note that, in FIG. 5A, each of the maximum blood pressure display region 501, the minimum blood pressure display region 502, and the pulse rate display region 503 is indicated by an oval of a broken line, but this is for easy understanding, and such an oval of the broken line is not displayed on an actual display screen. Note that the display 50 may include an organic electro luminescence (EL) display and may include a light emitting diode (LED).

In this example, the operation unit 52 illustrated in FIG. 1 includes a measurement switch 52A for receiving an instruction to start and stop measurement of a blood pressure, a memory switch 52B for calling a past measurement result, and a communication switch 52C for receiving an instruction to transmit data related to a measured value to the outside. These switches 52A, 52B, and 52C input operation signals respectively according to instructions from the user (for example, a subject) to the control unit 110.

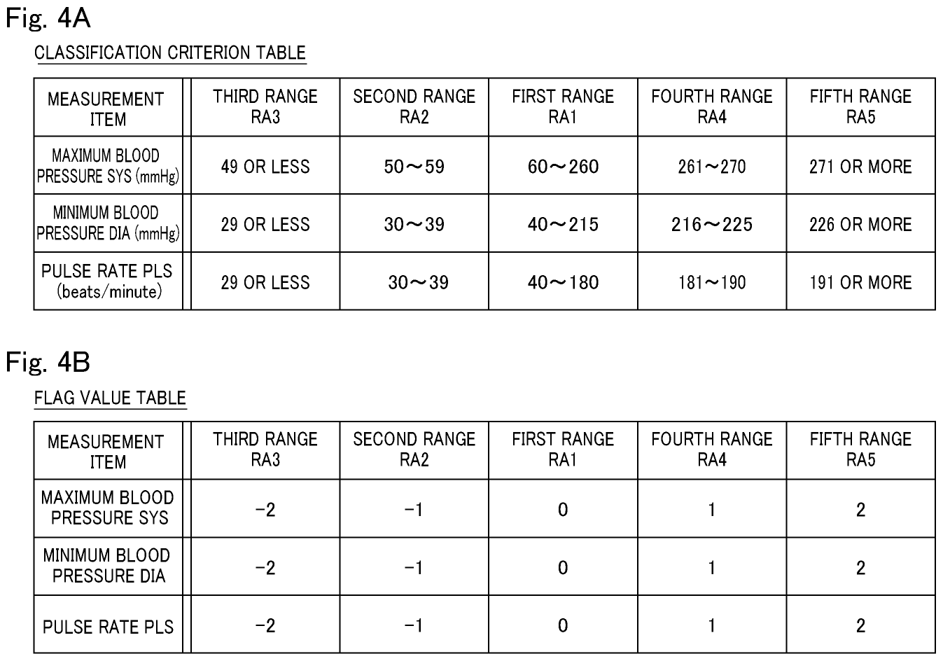

The memory 51 stores data of a program for controlling the sphygmomanometer 100, setting data for setting various functions of the sphygmomanometer 100, data of measured values, and the like. Further, the memory 51 is used as a work memory or the like when a program is executed. Further, in this example, the memory 51 stores a classification criterion table illustrated in FIG. 4A and a flag value table illustrated in FIG. 4B to be described later. Note that the flag value table is not used when the sphygmomanometer 100 operates alone, but is used when the sphygmomanometer 100 operates in a medical information management system to be described later.

The control unit 110 includes a central processing unit (CPU) as a processor, and controls entire operation of the sphygmomanometer 100. Specifically, the control unit 110 acts as a pressure control unit according to a program for controlling the sphygmomanometer 100 stored in the memory 51, and performs control to drive the pump 32 and the valve 33 as pressure devices according to operation signals from the operation unit 52. Further, the control unit 110 acts as a measured value acquisition unit, a determination unit, and a display processing unit, and in this example, calculates a blood pressure value (the maximum blood pressure SYS and the minimum blood pressure DIA) by a publicly-known oscillometric method based on outputs of the pressure sensor 31, and controls the display 50 and the memory 51. A specific method of blood pressure measurement will be described later. Further, the control unit 110 also calculates the pulse rate PLS in addition to a blood pressure value.

The pressure sensor 31 is a piezoresistance type pressure sensor in this example, detects a pressure (this is referred to as “cuff pressure Pc”) of the fluid bag 22 contained in the cuff 20 through the air pipe 38, and outputs an electric signal representing the cuff pressure Pc. The cuff pressure Pc includes a signal (pulse wave information) indicating a fluctuation component due to a pulse wave. The A/D conversion circuit 310 performs A/D conversion (analog-to-digital conversion) of a signal output from the pressure sensor 31, and inputs the signal to the control unit 110. The control unit 110 extracts pulse wave information from the signal representing the cuff pressure Pc and uses the pulse wave information for blood pressure calculation.

The pump 32, in this example, includes a commercially available rotary pump. The pump 32 is driven by the pump drive circuit 320 based on a control signal from the control unit 110. When the pump 32 is turned on, air is supplied to the fluid bag 22 included in the cuff 20 through the air pipe 38. By the above, a pressure (cuff pressure Pc) of the fluid bag 22 may be increased.

In this example, the valve 33 includes a normally-open type electromagnetic control valve, and is driven to open and close by the valve drive circuit 330 based on a control signal given from the control unit 110. The valve 33 is used to control a cuff pressure by discharging air in the fluid bag 22 through the air pipe 38 or filling the fluid bag 22 with air.

The power supply unit 53 supplies power to the control unit 110, the display 50, the memory 51, the pressure sensor 31, the pump 32, the valve 33, and other units in the main body 10.

When the communication switch 52C is turned on, a communication unit 90 transmits information (in this example, measured value data) from the control unit 110 to another device (for example, a server) via a network 900. Further, information from another device is received via the network 900 and tlansferred to the control unit 110.

The classification criterion table illustrated in FIG. 4A indicates a classification criterion that defines ranges into which a measured value of each of measurement items of the maximum blood pressure SYS, the minimum blood pressure DIA, and the pulse rate PLS should be classified. In this example, as shown in the top of the classification criterion table, for each measurement item, five ranges are set, the five range being a first range RA1 preset in a specification, a second range RA2 corresponding to a measured value that can be exhibited by a human, preset adjacent to a side lower than the first range RA1, a third range RA3 corresponding to an abnormal measured value that cannot be exhibited by a human, preset adjacent to a side lower than the second range RA2, a fourth range RA4 corresponding to a measured value that can be exhibited by a human, preset adjacent to a side higher than the first range RA1, and a fifth range RA5 corresponding to an abnormal measured value that cannot be exhibited by a human, preset adjacent to a side higher than the fourth range RA4. Here, a “human” refers to a general person, and may be a plurality of persons. A “human” may include a “subject”.

The first range RA1 is set in advance in a specification as an original measurement range. The first range RA1 may be substantially set by an instruction manual of a device or the like without limitation to a specification sheet.

The second range RA2 is preset adjacent to a side lower than the first range RA1 based on an empirical fact so as to include distribution of measured values that may be exhibited, for example, when a human is sleeping at night, when a human drinks alcohol and a blood vessel expands, when a medicine (for example, a hypotensive agent) taken by a human is too effective, and the like.

The fourth range RA4 is preset, based on an empirical fact, adjacent to a side higher than the first range RAI so as to include distribution of measured values that can be exhibited, for example, when a human is active in the daytime, when a human develops increase in a blood pressure with apnea syndrome during sleep, when a human develops increase in a blood pressure at the time of getting up, and the like.

The third range RA3 and the fifth range RA5 are preset based on an empirical fact as a range of abnormal measured values that cannot be exhibited by a human.

Hereinafter, first, second, . . . , and fifth ranges for the maximum blood pressure SYS will be referred to as RA1_SYS, RA2_SYS, . . . , and RA5_SYS. First, second, . . . , and fifth ranges for the minimum blood pressure DIA are referred to as RA1_DIA, RA2_DIA, . . . , and RA5_DIA. Further, first, second, . . . , and fifth ranges for the pulse rate PLS are referred to as RA1_PLS, RA2_PLS, . . . , and RA5_PLS. Here, the first range RA1 collectively refers to RA1_SYS, RA1_DIA, and RA1_PLS. The second range RA2 collectively refers to RA2_SYS, RA2_DIA, and RA2_PLS. Hereinafter, similarly, the fifth range RA5 collectively refers to RA5_SYS, RA5_DIA, and RA5_PLS.

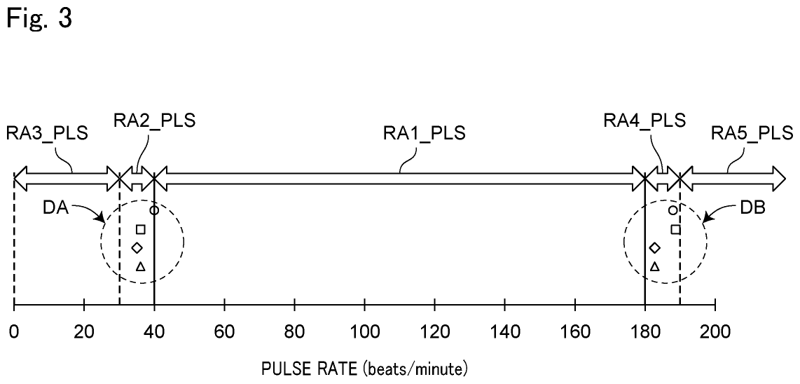

Specifically, as for the pulse rate PLS, for example, as illustrated in FIG. 3, the present inventor observed a measured value that can be exhibited by a human under various conditions such as at night or in the daytime, at rest or during activity, at the time of drinking or at the time of being sober, when medicine is taken or when taking medicine is stopped, and the like. Then, with respect to the first range RA1_PLS set in advance by a specification, the second range RA2_PLS adjacent to a side lower than the first range RA1 and the fourth range RA4_PLS adjacent to a side higher than the first range RA1 were obtained. In response, the third range RA3_PLS corresponding to an abnormal measured value that cannot be exhibited by a human was set adjacent to a side lower than the second range RA2. Further, the fifth range RA5_PLS corresponding to an abnormal measured value that cannot be exhibited by a human was set adjacent to a side higher than the fourth range RA4. As a result, as illustrated in a row of the pulse rate PLS in the classification criterion table of FIG. 4A, the first range RA1_PLS for the pulse rate PLS was set to 40 to 180 beats/min, the second range RA2_PLS was set to 30 to 39 beats/min, the third range RA3_PLS was set to 29 beats/min or less, the fourth range RA4_PLS was set to 181 to 190 beats/min, and the fifth range RA5_PLS was set to 191 beats/min or more.

Similarly, as indicated in a row of the maximum blood pressure SYS of the classification criterion table of FIG. 4A, the first range RA1_SYS for the maximum blood pressure SYS was set to 60 to 260 mmHg, the second range RA2_SYS was set to 50 to 59 mmHg, the third range RA3_SYS was set to 49 mmHg or less, the fourth range RA4_SYS was set to 261 to 270 mmHg, and the fifth range RA5_SYS was set to 271 mmHg or more.

Further, similarly, as illustrated in a row of the minimum blood pressure DIA in the classification criterion table of FIG. 4A, the first range RA1_DIA for the minimum blood pressure DIA was set to 40 to 215 mmHg, the second range RA2_DIA was set to 30 to 39 mmHg, the third range RA3_DIA was set to 29 mmHg or less, the fourth range RA4_DIA was set to 216 to 225 mmHg, and the fifth range RA5_DIA was set to 226 mmHg or more.

In this example, measured values of the maximum blood pressure SYS and the minimum blood pressure DIA are calculated and displayed in units of mmHg by being rounded off to the nearest whole number. In accordance with the above, the first, second, . . . , fifth ranges RA1_SYS, RA2_SYS, . . . , RA5_SYS with respect to the maximum blood pressure SYS, and the first, second, . . . , fifth ranges RA1_DIA, RA2_DIA, . . . , RA5_DIA with respect to the minimum blood pressure DIA are also set in units of mmHg by being rounded off to the nearest whole number. Further, a measured value of the pulse rate PLS is calculated and displayed in units of beats/minute by being rounded off to the nearest whole number. In accordance with the above, the first, second, . . . , and fifth ranges RA1_PLS, RA2_PLS, . . . , and RA5_PLS with respect to the pulse rate PLS are also set in units of beats/minute by being rounded off to the nearest whole number.

### Blood Pressure Measurement and Display of Measurement Result

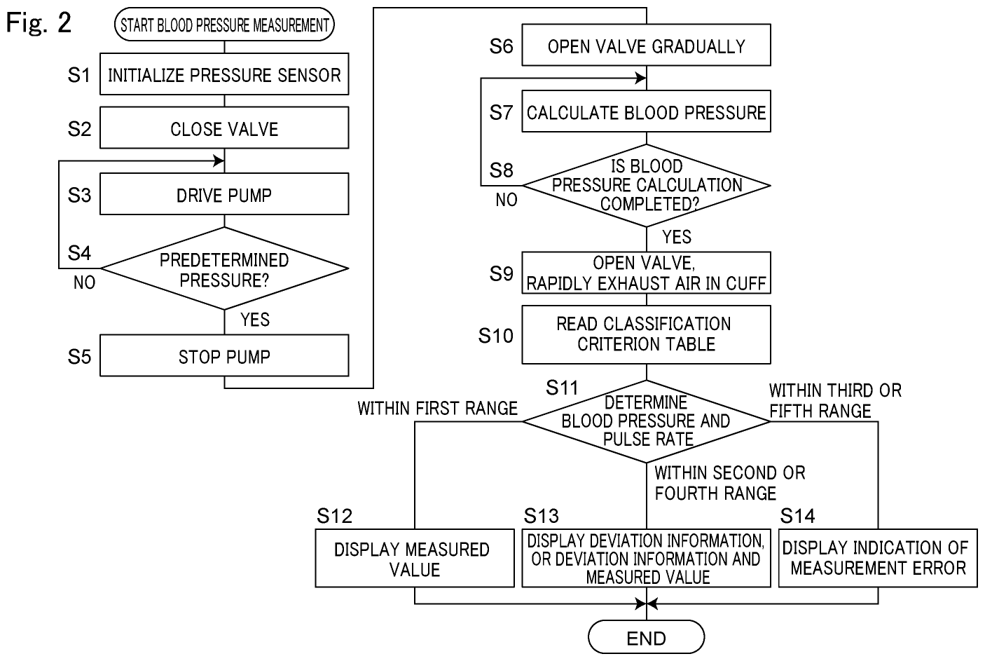

FIG. 2 illustrates a flow of processing in which the sphygmomanometer 100 alone measures a blood pressure of a subject and displays the measured maximum blood pressure SYS, minimum blood pressure DIA, and pulse rate PLS on the display screen of the display 50.

In an attached state in which the cuff 20 is attached to a measurement target site (for example, an upper arm of a subject), when the user (in this example, the subject) gives an instruction to start measurement using the measurement switch 52A provided on the main body 10, the control unit 110 performs initialization (step S1 in FIG. 2). Specifically, the control unit 110 initializes a processing memory area, stops the pump 32, and performs 0-mmHg adjustment (sets the atmospheric pressure to 0 mmHg) of the pressure sensor 31 in a state where the valve 33 is opened.

Next, the control unit 110 acts as a pressure control unit to close the valve 33 (step S2), to drive the pump 32 via the pump drive circuit 320, and to start pressurization of the cuff 20 (step S3). That is, the control unit 110 supplies air from the pump 32 to (the fluid bag 22 contained in) the cuff 20 through the air pipe 38. At the same time, the pressure sensor 31 acts as a pressure detection unit to detect pressure (cuff pressure Pc) of the fluid bag 22 through the air pipe 38. The control unit 110 controls a pressurization rate by the pump 32 based on output of the pressure sensor 31. By this pressurization, an artery passing through the measurement target site is pressed and made ischemic.

Next, the control unit 110 determines whether or not the cuff pressure Pc reaches a predetermined pressure based on output of the pressure sensor 31 (step S4). The “predetermined pressure” is preset to a pressure value sufficiently higher than an assumed blood pressure value of the subject. In this example, the predetermined pressure is preset to be a blood pressure value (maximum blood pressure SYS) of the subject measured last time plus 40 mmHg. When the cuff pressure Pc reaches the predetermined pressure (YES in step S4), the control unit 110 stops the pump 32 (step S5).

Subsequently, the control unit 110 gradually opens the valve 33 (step S6). By the above, the cuff pressure Pc is reduced at a substantially constant speed. Here, a pulse wave signal (fluctuation component) as pulse wave information by a pulse wave is superimposed on the cuff pressure Pc detected by the pressure sensor 31 through the air pipe 38.

In this depressurization process, the control unit 110 acts as a blood pressure calculation unit, extracts a pulse wave signal (fluctuation component) superimposed on the cuff pressure Pc, and attempts to calculate a blood pressure value (the maximum blood pressure SYS and the minimum blood pressure DIA) by a publicly-known oscillometric method in this example based on a pulse wave signal acquired at this time point (step S7). Further, in this example, the control unit 110 calculates the pulse rate PLS on the basis of the pulse wave signal. In this manner, the control unit 110 acts as a measured value acquisition unit to acquirs a measured value for each measurement item for three measurement items of the maximum blood pressure SYS, the minimum blood pressure DIA, and the pulse rate PLS in this example.

In a case where a blood pressure value and a pulse rate cannot be calculated yet due to a shortage of data (NO in step S8), the control unit 110 repeats the processing of steps S7 to S8 until a blood pressure value and a pulse rate can be calculated.

When a blood pressure value and a pulse rate can be calculated in this manner (YES in step S8), the control unit 110 acts as a pressure control unit to perform control to open the valve 33 and rapidly exhaust air in the cuff 20 (fluid bag 22) (step S9).

Note that calculation of a blood pressure value and a pulse rate may be performed in a pressurization process from start of pressurization of the cuff 20 until the cuff pressure Pc reaches the predetermined pressure, instead of being performed in the depressurization process as in the above example.

Subsequently, in step S10 of FIG. 2, the control unit 110 reads the classification criterion table of FIG. 4A stored in the memory 51. Then, in step S11 of FIG. 2, with referring to the classification criterion table, the control unit 110 acts as a determination unit to determine which one of the first, second, . . . , and fifth ranges RA1, RA2, . . . , and RA5 the acquired measured value is classified into for each measurement item.

When the measured value is classified into the first range RA1 in step S11, the control unit 110 acts as a display processing unit to perform processing of displaying the measured value on the display screen of the display 50 as illustrated in step S12.

Alternatively, when the measured value is classified into the second range RA2 or the fourth range RA4 in step S11, the control unit 110 acts as a display processing unit to perform processing of displaying, on the display screen of the display 50, deviation information indicating that the measured value is classified into the second range RA2 or the fourth range RA4, or the measured value in addition to the deviation information as illustrated in step S13.

Alternatively, when the measured value is classified into the third range RA3 or the fifth range RA5 in step S11, the control unit 110 acts as a display processing unit to display an indication of measurement error with displaying neither the deviation information nor the measured value on the display screen of the display 50, as illustrated in step S14.

In this example, the processing in steps S12 to S14 is performed for each measurement item of the maximum blood pressure SYS, the minimum blood pressure DIA, and the pulse rate PLS (at the same time, the acquired maximum blood pressure SYS, minimum blood pressure DIA, and pulse rate PLS are stored in the memory 51).

For example, in the example of FIG. 5A, a measured value of the maximum blood pressure SYS is 124 mmHg, which falls within the first range RA1_SYS (60 to 260 mmHg) for the maximum blood pressure SYS. A measured value of the minimum blood pressure DIA is 74 mmHg, which falls within the first range RA1_DIA (40 to 215 mmHg) for the minimum blood pressure DIA. Further, a measured value of the pulse rate PLS is 61 beats/minute, which falls within the first range RA1_PLS (40 to 180 beats/minute) for the pulse rate PLS. In this case, as illustrated in FIG. 5A, on the display screen of the display 50, the measured value “124” of the maximum blood pressure SYS, the measured value “74” of the minimum blood pressure DIA, and the measured value “61” of the pulse rate PLS are displayed in the right side portion 501b of the maximum blood pressure display region 501, the right side portion 502b of the minimum blood pressure display region 502, and the right side portion 503b of the pulse rate display region 503, respectively. By the above, a subject can know a measured value for each measurement item.

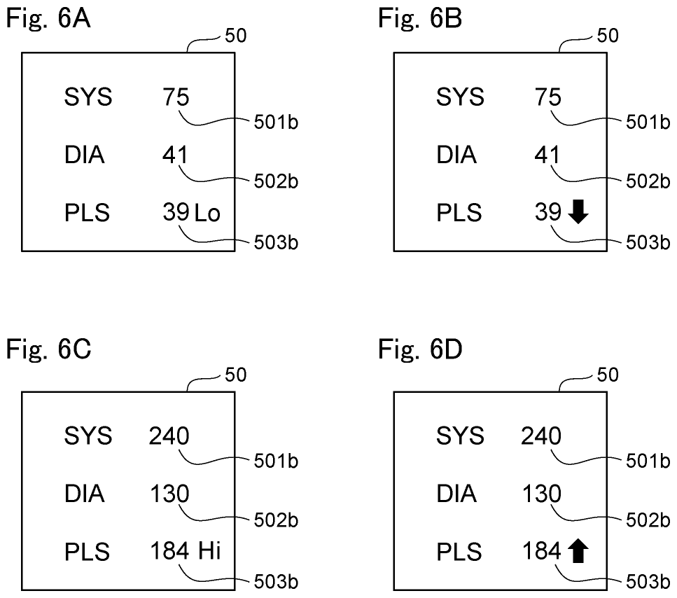

In the examples of FIGS. 5B, 5C, 6A, and 6B, a measured value of the maximum blood pressure SYS is 75 mmHg, which falls within the first range RA1_SYS (60 to 260 mmHg) for the maximum blood pressure SYS. A measured value of the minimum blood pressure DIA is 41 mmHg, which falls within the first range RA1_DIA (40 to 215 mmHg) for the minimum blood pressure DIA. On the other hand, a measured value of the pulse rate PLS is 39 beats/minute, which falls within the second range RA2_PLS (30 to 39 beats/minute) for the pulse rate PLS. In this case, as illustrated in FIGS. 5B, 5C, 6A, and 6B, on the display screen of the display 50, the measured value “75” of the maximum blood pressure SYS and the measured value “41” of the minimum blood pressure DIA are displayed in the right side portion 501b of the maximum blood pressure display region 501 and the right side portion 502b of the minimum blood pressure display region 502, respectively. On the other hand, as illustrated in FIGS. 5B and 5C, deviation information indicating that a measured value of the pulse rate PLS is classified into the second range RA2_PLS is displayed in the right side portion 503b of the pulse rate display region 503, for example. Here, in the example of FIG. 5B, the deviation information is represented by a character string “Lo”. In the example of FIG. 5C, the deviation information is indicated by a downward arrow mark. By the above, a subject can know the measured value of the maximum blood pressure SYS and the measured value of the minimum blood pressure DIA, and can know that the measured value of the pulse rate PLS deviates to the second range RA2_PLS (a range that is lower than an original measurement range and corresponds to a measured value that can be exhibited by a human). Alternatively, as illustrated in FIGS. 6A and 6B, the measured value “39” of the pulse rate PLS and deviation information indicating that the measured value of the pulse rate PLS is classified into the second range RA2_PLS may be displayed together in the right side portion 503b of the pulse rate display region 503. In the example of FIG. 6A, a character string “39 Lo” obtained by combining the measured value “39” of the pulse rate PLS with the deviation information “Lo” is displayed. In the example of FIG. 6B, the measured value “39” of the pulse rate PLS and a downward arrow mark as deviation information are displayed together. By the above, a subject can know the measured value of the maximum blood pressure SYS and the measured value of the minimum blood pressure DIA, and can know that the measured value of the pulse rate PLS deviates to the second range RA2_PLS and the measured value deviating to the second range RA2_PLS.

In the examples of FIGS. 5D, 5E, 6C, and 6D, a measured value of the maximum blood pressure SYS is 240 mmHg, which falls within the first range RA1_SYS (60 to 260 mmHg) for the maximum blood pressure SYS. A measured value of the minimum blood pressure DIA is 130 mmHg, which falls within the first range RA1_DIA (40 to 215 mmHg) for the minimum blood pressure DIA. On the other hand, a measured value of the pulse rate PLS is 184 beats/minute, which falls within the fourth range RA4_PLS (181 to 190 beats/minute) for the pulse rate PLS. In this case, as illustrated in FIGS. 5D, 5E, 6C, and 6D, on the display screen of the display 50, the measured value “240” of the maximum blood pressure SYS and the measured value “130” of the minimum blood pressure DIA are displayed in the right side portion 501b of the maximum blood pressure display region 501 and the right side portion 502b of the minimum blood pressure display region 502, respectively. On the other hand, as illustrated in FIGS. 5D and 5E, deviation information indicating that a measured value of the pulse rate PLS is classified into the fourth range RA4_PLS is displayed in the right side portion 503b of the pulse rate display region 503, for example. Here, in the example of FIG. 5D, the deviation information is represented by a character string “Hi”. In the example of FIG. 5E, the deviation information is indicated by an upward arrow mark. By the above, a subject can know the measured value of the maximum blood pressure SYS and the measured value of the minimum blood pressure DIA, and can know that the measured value of the pulse rate PLS deviates to the fourth range RA4_PLS (a range that is higher than an original measurement range and corresponds to a measured value that can be exhibited by a human). Alternatively, as illustrated in FIGS. 6C and 6D, the measured value “184” of the pulse rate PLS and the deviation information indicating that the measured value is classified into the fourth range RA4_PLS may be displayed together in the right side portion 503b of the pulse rate display region 503. In the example of FIG. 6C, a character string “184Hi” obtained by combining the measured value “184” of the pulse rate PLS with the deviation information “Hi” is displayed. In the example of FIG. 6D, the measured value “184” of the pulse rate PLS and an upward arrow mark as deviation information are displayed together. By the above, a subject can know the measured value of the maximum blood pressure SYS and the measured value of the minimum blood pressure DIA, and can know that the measured value of the pulse rate PLS deviates to the fourth range RA4_PLS and the measured value deviating to the fourth range RA4_PLS.

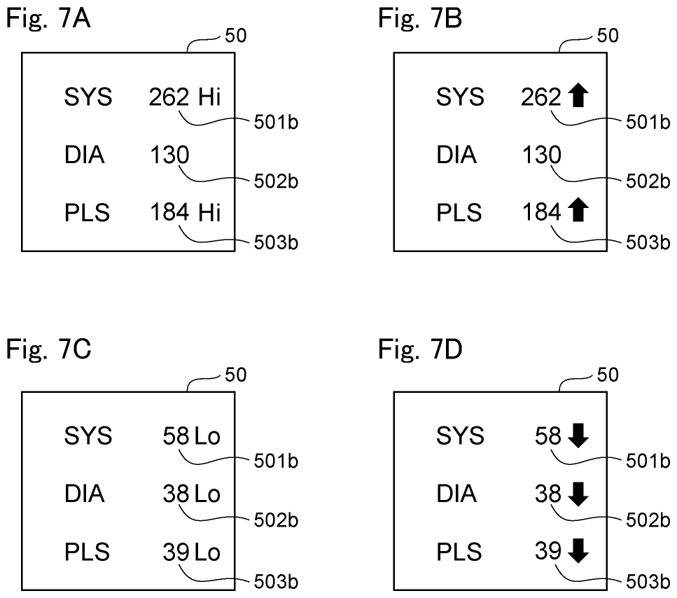

In the examples of FIGS. 7A and 7B, a measured value of the maximum blood pressure SYS is 262 mmHg, which falls within the fourth range RA4_SYS (261 to 270 mmHg) for the maximum blood pressure SYS. A measured value of the minimum blood pressure DIA is 130 mmHg, which falls within the first range RA1_DIA (40 to 215 mmHg) for the minimum blood pressure DIA. A measured value of the pulse rate PLS is 184 beats/minute, which falls within the fourth range RA4_PLS (181 to 190 beats/minute) for the pulse rate PLS. In this case, as illustrated in FIG. 7A, on the display screen of the display 50, for example, a character string “262Hi” obtained by combining the measured value “262” of the maximum blood pressure SYS and the deviation information “Hi” is displayed in the right side portion 501b of the maximum blood pressure display region 501. The measured value “130” of the minimum blood pressure DIA is displayed in the right side portion 502b of the minimum blood pressure display region 502. Further, for example, a character string “184Hi” obtained by combining the measured value “184” for the pulse rate PLS with the deviation information “Hi” is displayed in the right side portion 503b of the pulse rate display region 503. Alternatively, as illustrated in FIG. 7B, for example, the measured value “262” of the maximum blood pressure SYS and the upward arrow mark as deviation information are displayed together in the right side portion 501b of the maximum blood pressure display region 501. The measured value “130” of the minimum blood pressure DIA is displayed in the right side portion 502b of the minimum blood pressure display region 502. Further, for example, the measured value “184” for the pulse rate PLS and the upward arrow mark as deviation information are displayed together in the right side portion 503b of the pulse rate display region 503. By the above, a subject can know the measured value of the minimum blood pressure DIA, and can also know that the measured value of the maximum blood pressure SYS and the measured value of the pulse rate PLS deviate to the fourth ranges RA4_SYS and RA4_PLS, respectively, and the measured values deviating to the fourth ranges RA4_SYS and RA4_PLS. Note that, alternatively, the configuration may be such that the measured value with respect to the maximum blood pressure SYS and the measured value for the pulse rate PLS are not displayed, and only deviation information (for example, the character string “Hi” or an upward arrow mark) indicating that the measured values are classified into the fourth ranges RA4_SYS and RA4_PLS are displayed in the right side portion 501b of the maximum blood pressure display region 501 and the right side portion 503b of the pulse rate display region 503.

In the examples of FIGS. 7C and 7D, a measured value of the maximum blood pressure SYS is 58 mmHg, which falls within the second range RA2_SYS (50 to 59 mmHg) for the maximum blood pressure SYS. A measured value of the minimum blood pressure DIA is 38 mmHg, which falls within the second range RA2_DIA (30 to 39 mmHg) for the minimum blood pressure DIA. A measured value of the pulse rate PLS is 39 beats/minute, which falls within the second range RA2_PLS (30 to 39 beats/minute) for the pulse rate PLS. In this case, as illustrated in FIG. 7C, on the display screen of the display 50, for example, a character string “58Lo” obtained by combining the measured value “58” of the maximum blood pressure SYS and the deviation information “Lo”, a character string “38Lo” obtained by combining the measured value “38” of the minimum blood pressure DIA and the deviation information “Lo”, and a character string “39Lo” obtained by combining the measured value “39” of the pulse rate PLS and the deviation information “Lo” are displayed in the right side portion 501b of the maximum blood pressure display region 501, the right side portion 502b of the minimum blood pressure display region 502, and the right side portion 503b of the pulse rate display region 503, respectively. Alternatively, as illustrated in FIG. 7D, display in which the measured value “58” of the maximum blood pressure SYS and the downward arrow mark as the deviation information are combined, display in which the measured value “38” of the minimum blood pressure DIA and the downward arrow mark as the deviation information are combined, and display in which the measured value “39” of the pulse rate PLS and the downward arrow mark as the deviation information are combined are performed in the right side portion 501b of the maximum blood pressure display region 501, the right side portion 502b of the minimum blood pressure display region 502, and the right side portion 503b of the pulse rate display region 503, respectively. By the above, a subject can know that the measured value of the maximum blood pressure SYS, the measured value of the minimum blood pressure DIA, and the measured value of the pulse rate PLS deviate to the second ranges RA2_SYS, RA2_DIA, and RA2_PLS, respectively, and the measured values deviating to the second ranges RA2_SYS, RA2_DIA, and RA2_PLS. Note that, alternatively, the configuration may be such that the measured value with respect to the maximum blood pressure SYS, the measured value with respect to the minimum blood pressure DIA, and the measured value for the pulse rate PLS are not displayed, and only deviation information (for example, the character string “Lo” or the downward arrow mark) indicating that the measured values are classified into the second ranges RA2_SYS, RA2_DIA, and RA2_PLS are displayed in the right side portion 501b of the maximum blood pressure display region 501, the right side portion 502b of the minimum blood pressure display region 502, and the right side portion 503b of the pulse rate display region 503.

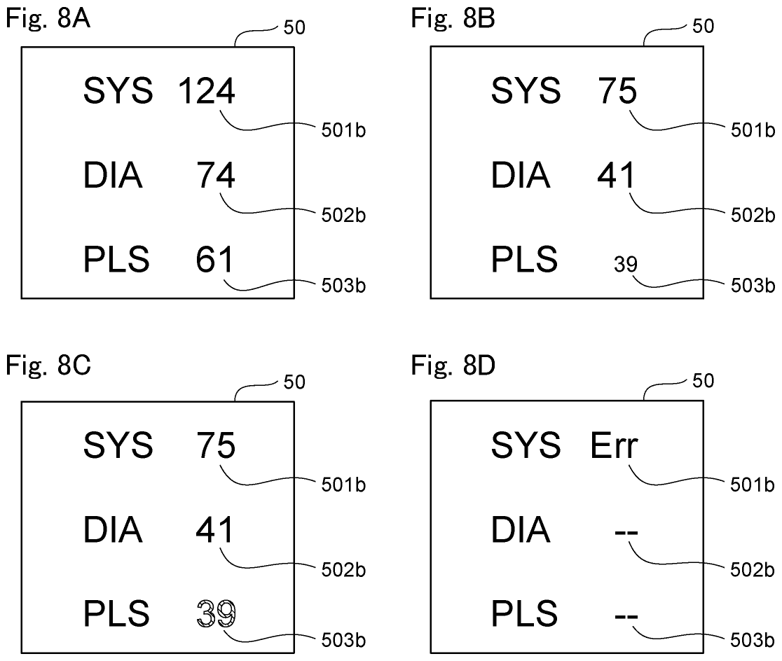

In the above-described examples of FIGS. 5B to 5E, 6A to 6D, and 7A to 7D, the deviation information is displayed as the character string “Lo” or “Hi”, or a downward arrow mark or an upward arrow mark, but the present invention is not limited to this. For example, in a case where all of a measured value of the maximum blood pressure SYS, a measured value of the minimum blood pressure DIA, and a measured value of the pulse rate PLS fall within the first range RA1, it is assumed that, as illustrated in FIG. 8A, the measured value of the maximum blood pressure SYS, the measured value of the minimum blood pressure DIA, and the measured value of the pulse rate PLS are displayed in the first mode in the right side portion 501b of the maximum blood pressure display region 501, the right side portion 502b of the minimum blood pressure display region 502, and the right side portion 503b of the pulse rate display region 503, respectively, on the display screen of the display 50. Here, the first mode refers to a predetermined size, thickness, and color in which a measured value is displayed. On the other hand, for example, in a case where a measured value of the maximum blood pressure SYS and a measured value of the minimum blood pressure DIA fall within the first range RA1 but a measured value of the pulse rate PLS falls within the second range RA2, as illustrated in FIGS. 8B and 8C, the measured value of the maximum blood pressure SYS (“75” in this example) and the measured value of the minimum blood pressure DIA (“41” in this example) may be displayed in the first mode in the right side portion 501b of the maximum blood pressure display region 501 and the right side portion 502b of the minimum blood pressure display region 502, respectively, and the measured value of the pulse rate PLS (“39” in this example) may be displayed in the right side portion 503b of the pulse rate display region 503 in the second mode distinguishable from the first mode. Here, the second mode refers to a mode in which at least one of a size, a thickness, and a color in which a measured value is displayed is different from that in the first mode. In the example of FIG. 8B, as the second mode, the measured value “39” of the pulse rate PLS is displayed with a smaller size than a size at which a measured value is displayed in the first mode and a smaller thickness. In the example of FIG. 8C, as the second mode, the measured value “39” of the pulse rate PLS is displayed in a color (orange in an actual product, but for convenience of illustration, represented by black hatching on the white background) different from a color (black in this example) of a measured value in the first mode.

In a case where any one of a measured value of the maximum blood pressure SYS, a measured value of the minimum blood pressure DIA, and a measured value of the pulse rate PLS falls within the third range RA3 or the fifth range RA5, an indication of measurement error (in this example, the character string “Err”) is displayed on the display screen of the display 50 as illustrated in FIG. 8D, similarly as described in the conventional example. For example, it is assumed that a measured value of the maximum blood pressure SYS is 124 mmHg and falls within the first range RA1_SYS (60 to 260 mmHg) for the maximum blood pressure SYS, and a measured value of the minimum blood pressure DIA is 74 mmHg and falls within the first range RA1_DIA (40 to 215 mmHg) for the minimum blood pressure DIA. Even in this case, for example, when a measured value of the pulse rate PLS is 29 beats/minute and falls within the third range RA3_PLS (29 beats/minute or less) for the pulse rate PLS, not only the measured value of the pulse rate PLS but also the measured value of the maximum blood pressure SYS and the measured value of the minimum blood pressure DIA are not displayed on the display screen of the display 50, and the indicaton of measurement error as illustrated in FIG. 8D is displayed. By the above, a subject can know that a measurement error occurs. Note that, in the example of FIG. 8D, the character string “Err” is displayed on the right side of the character string “SYS” (the right side portion 501b of the maximum blood pressure display region 501), but the position of the character string “Err” has no meaning.

As described above, according to the sphygmomanometer 100, for each measurement item, even in a case where an acquired measured value deviates from the first range RA1 to the lower side (the second range RA2) or the higher side (the fourth range RA4), information on a blood pressure of a subject (deviation information or a measured value in addition to the deviation information) can be displayed. Therefore, convenience of a subject as the user can be enhanced. In particular, in a case where the deviation information is displayed as a mark (for example, a downward arrow or an upward arrow), a subject can intuitively recognize that a measured value deviates to the second range RA2 or fourth range RA4 by viewing the mark. Similarly, in a case where the deviation information is displayed in the second mode (for example, a smaller size, a thinner thickness, or a different color) distinguishable from the first mode, a subject can intuitively recognize that a measured value deviates to the second range RA2 or fourth range RA4 by viewing a measured value displayed in the second mode. Therefore, convenience of a subject as the user can be further enhanced.

### Second Embodiment

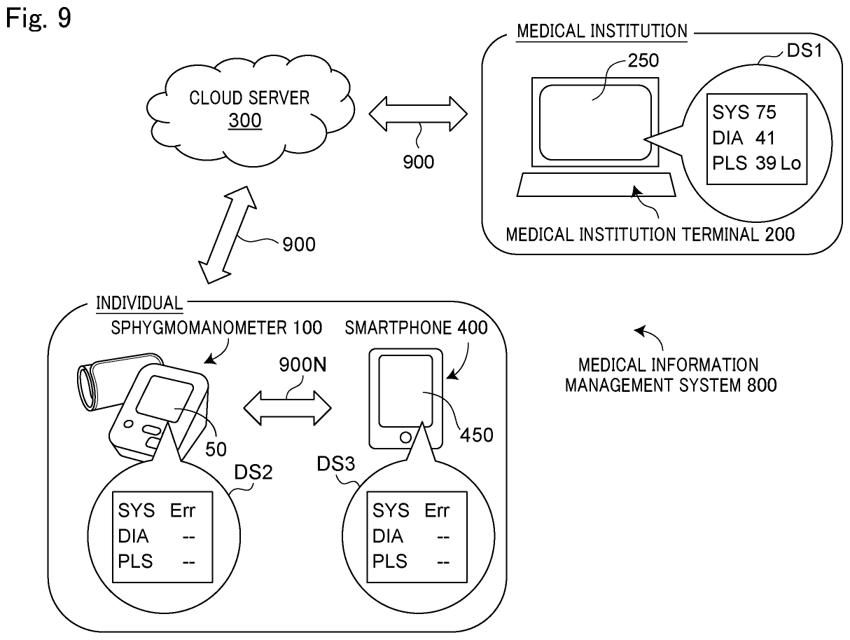

FIG. 9 illustrates a schematic configuration of a medical information management system 800 as another embodiment to which the blood-pressure-related information display device of the present invention is applied. The system 800 includes the sphygmomanometer 100 described above, a smartphone 400 communicably connected to the sphygmomanometer 100 via a short-range communication line 900N, a cloud server 300 communicably connected to the sphygmomanometer 100 and the smartphone 400 via a network (in this example, the Internet, which similarly applies hereinafter) 900, and a medical institution terminal 200 communicably connected to the cloud server 300 via the network 900. In this example, the sphygmomanometer 100 and the smartphone 400 are intended to be managed and used by an individual subject. The short-range communication line 900N refers to Wi-Fi (registered trademark), Bluetooth (registered trademark), or the like. The medical institution terminal 200 is intended to be managed and used not by a subject but by a medical personnel (a doctor, a nurse, or the like) engaged in a medical institution (a hospital, a clinic, or the like). The sphygmomanometer 100 and the smartphone 400, the cloud server 300, and the medical institution terminal 200 are spatially separated from each other.

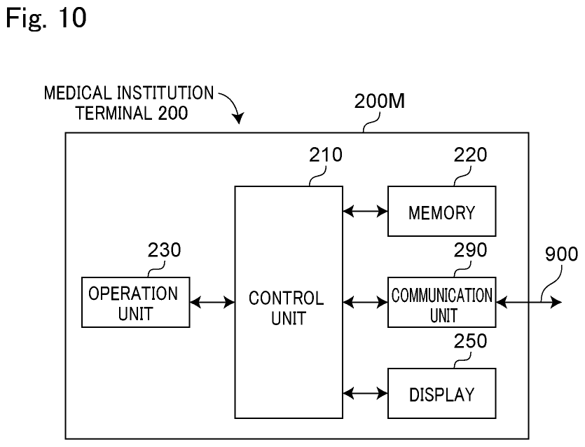

As illustrated in FIG. 10, the medical institution terminal 200 includes a main body 200M, a control unit 210, a memory 220, an operation unit 230, a display 250, and a communication unit 290 mounted on the main body 200M. The medical institution terminal 200 includes a commercially available notebook personal computer, and has application software (computer program) installed so as to perform processing to be described later.

The control unit 210 includes a central processing unit (CPU) and an auxiliary circuit of the CPU, controls each unit of the medical institution terminal 200, and executes processing to be described later according to a program and data stored in the memory 220. That is, data input from the operation unit 230 and the communication unit 290 is processed, and the processed data is stored in the memory 220, displayed on the display 250, or output from the communication unit 290.

The memory 220 includes a random access memory (RAM) used as a work area necessary for the control unit 210 to execute a program, and a read only memory (ROM) for storing a basic program to be executed by the control unit 210. Further, a semiconductor memory (memory card, solid state drive (SSD)) or the like may be used as a storage medium of an auxiliary storage device for assisting a storage area of the memory 220.

In this example, the operation unit 230 includes a keyboard and a mouse, and typically inputs operation signals indicating operations by a medical personnel (in this example, a doctor) to the control unit 210. Further, the operation unit 230 may include another operation device such as a touch panel instead of or in addition to a keyboard and a mouse.

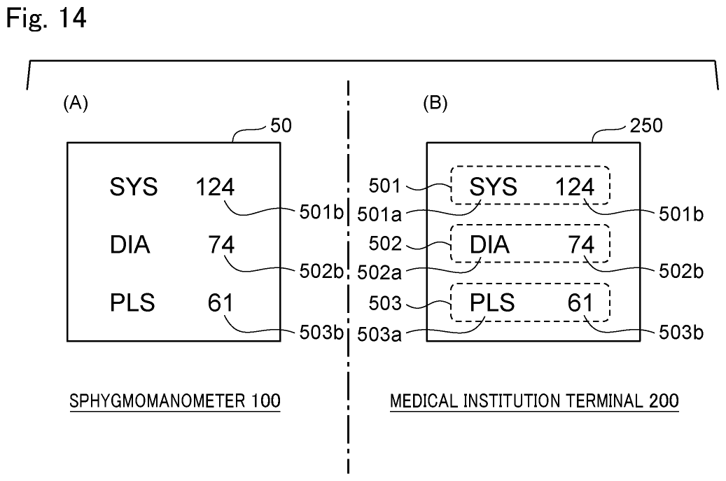

The display 250 includes, for example, a liquid crystal display (LCD), an electro luminescence (EL) display, or the like, and includes a display screen. The display 250 is controlled by the control unit 210 to display predetermined video on the display screen. In this example, by the application software described above, as illustrated in FIG. 14, portion (B), the display screen of the display 250 is provided with, in order from the top, a maximum blood pressure display region 501 for displaying the maximum blood pressure SYS (systolic blood pressure, units; mmHg), a minimum blood pressure display region 502 for displaying the minimum blood pressure DIA (diastolic blood pressure, units; mmHg), and a pulse rate display region 503 for displaying the pulse rate PLS (units; beat/minute) (for the sake of simplicity, these are represented by the same reference numerals as the display regions in the display 50 of the sphygmomanometer 100). The character strings “SYS”, “DIA”, and “PLS” are used in the left side portion 501a of the maximum blood pressure display region 501, the left side portion 502a of the minimum blood pressure display region 502, and the left side portion 503a of the pulse rate display region 503, respectively, similarly as in the display 50 of the sphygmomanometer 100. In each of the right side portion 501b of the maximum blood pressure display region 501, the right side portion 502b of the minimum blood pressure display region 502, and the right side portion 503b of the pulse rate display region 503, a measured value to be processed by the application software is normally displayed. Note that, in FIG. 14, portion (B), each of the maximum blood pressure display region 501, the minimum blood pressure display region 502, and the pulse rate display region 503 is indicated by an oval of a broken line, but this is for easy understanding, and such an oval of the broken line is not displayed on an actual display screen.

In this example, the communication unit 290 receives information on a

measured value (blood pressure value, pulse rate) of a subject from the cloud server 300 via the network 900. Further, the communication unit 290 transmits information from the control unit 210 to the cloud server 300 via the network 900.

### Blood Pressure Measurement and Display of Measurement Result

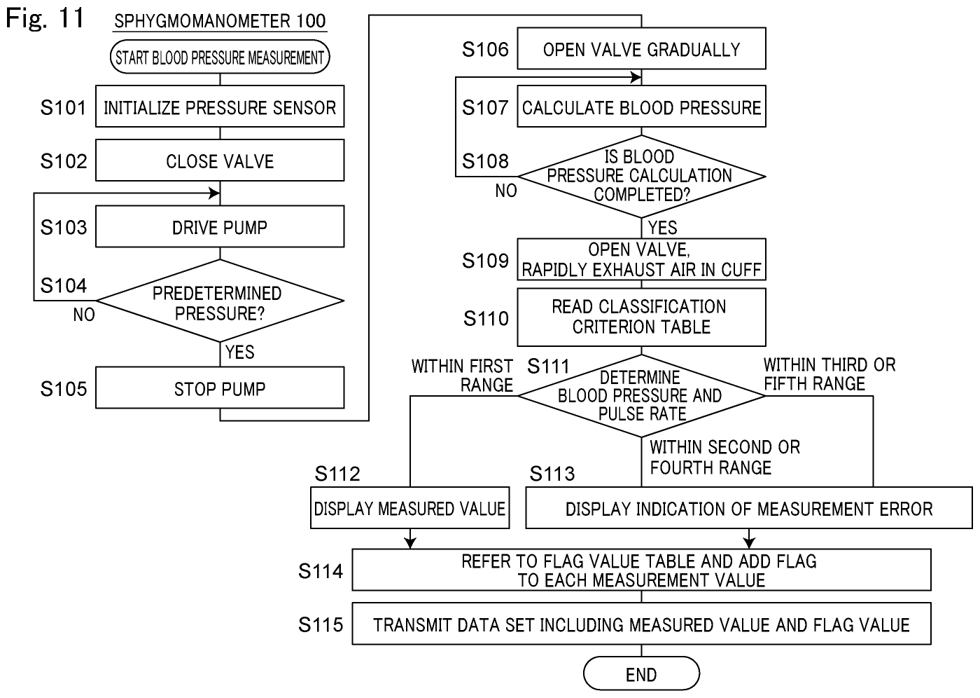

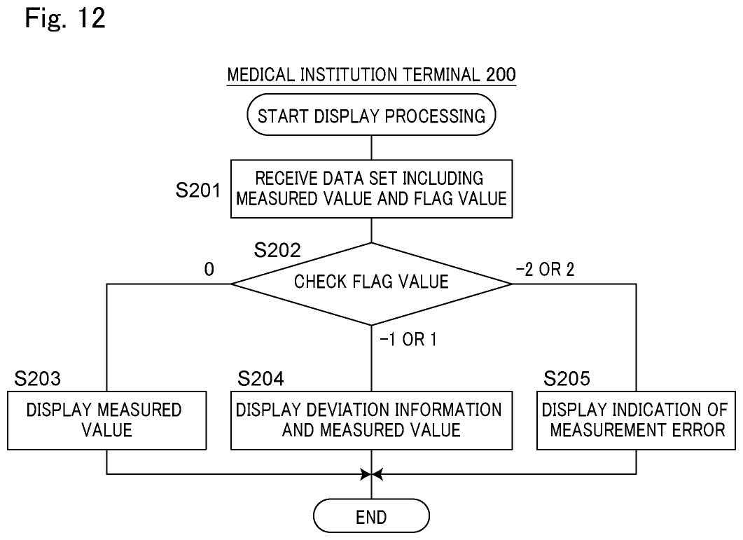

FIG. 11 illustrates an operation flow of the sphygmomanometer 100 included in the system 800, and FIG. 12 illustrates an operation flow of the medical institution terminal 200 cooperating with the operation flow of FIG. 11 of the sphygmomanometer 100.

When a subject instructs to start measurement with the measurement switch 52A provided on the main body 10 in an attached state where the cuff 20 of the sphygmomanometer 100 is attached to a measurement target site (for example, an upper arm of the subject), the control unit 110 of the sphygmomanometer 100 performs exactly the same processing as steps S1 to S9 of FIG. 2 to acquire a measured value for each of three measurement items of the maximum blood pressure SYS, the minimum blood pressure DIA, and the pulse rate PLS as illustrated in steps S101 to S109 of FIG. 11.

Subsequently, in step S110 of FIG. 11, the control unit 110 reads the classification criterion table of FIG. 4A stored in the memory 51. Then, in step S111 of FIG. 11, with referring to the classification criterion table, the control unit 110 acts as a determination unit to determine which one of the first, second, . . . , and fifth ranges RA1, RA2, . . . , and RA5 the acquired measured value is classified into for each measurement item.

When the measured value is classified into the first range RA1 in step S111, the control unit 110 acts as a second display processing unit to perform processing of displaying the measured value on the display screen (acting as a second display screen in the present embodiment, which similarly applies hereinafter) of the display 50 as illustrated in step S112.

Alternatively, when the measured value is classified into the second range RA2 or the fourth range RA4, or the third range RA3 or the fifth range RA5 in step S111 of FIG. 11, the control unit 110 acts as a second display processing unit to display an indication of measurement error with displaying neither the deviation information nor the measured value on the display screen of the display 50, as illustrated in step S113.

In this example, the processing in steps S112 to S113 is performed for each measurement item of the maximum blood pressure SYS, the minimum blood pressure DIA, and the pulse rate PLS (at the same time, the acquired maximum blood pressure SYS, minimum blood pressure DIA, and pulse rate PLS are stored in the memory 51). A display example on the display screen of the display 50 will be described later.

Next, in step S114 of FIG. 11, the control unit 110 acts as a flag adding unit to refer to the flag value table of FIG. 4B stored in the memory 51, and to add a flag having an integer value from −2 to 2 to the measured value according to which one of the first, second, . . . , and fifth ranges RA1, RA2, . . . , and RA5 the measured value is classified into. The flag table of FIG. 4B indicates a value of a flag to be added according to which one of the first, second, . . . , and fifth ranges RA1, RA2, . . . , RA5 the measured value is classified into for each measurement item. For example, the flag value is set to 0 when a measured value of the maximum blood pressure SYS is classified into the first range RA1, the flag value is set to −1 when the measured value is classified into the second range RA2, the flag value is set to −2 when the measured value is classified into the third range RA3, the flag value is set to 1 when the measured value is classified into the fourth range RA4, and the flag value is set to 2 when the measured value is classified into the fifth range RA5. A value of a flag to be added is similar for a measured value of the minimum blood pressure DIA and a measured value of the pulse rate PLS.

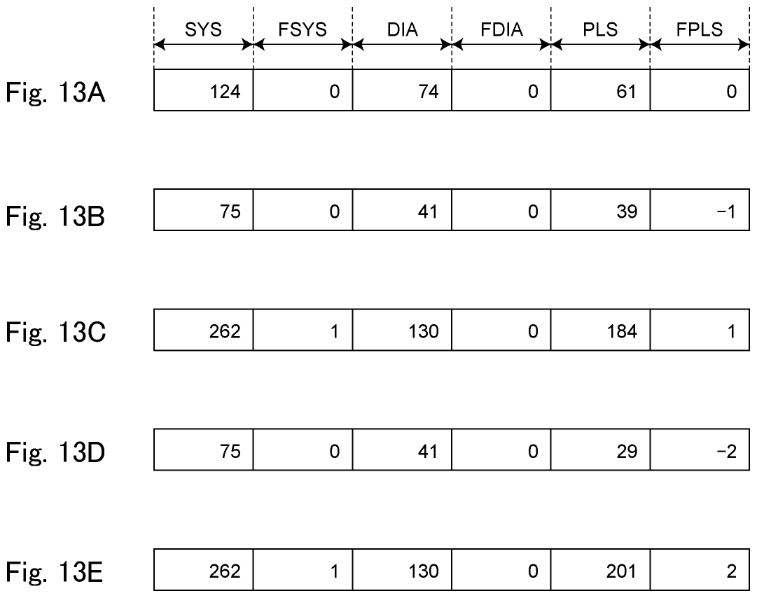

By the above, for example, as illustrated in FIGS. 13A to 13E, the control unit 110 creates a data set having a format in which a measured value of the maximum blood pressure SYS, a flag value FSYS for the measured value, a measured value of the minimum blood pressure DIA, a flag value FDIA for the measured value, a measured value of the pulse rate PLS, and a flag value FPLS for the measured value are arranged in this order as information on measured values of a subject.

For example, in the example of FIG. 13A, since a measured value 124 mmHg of the maximum blood pressure SYS falls within the first range RA1_SYS (60 to 260 mmHg) for the maximum blood pressure SYS, the flag value FSYS for the measured value is 0. Since a measured value 74 mmHg of the minimum blood pressure DIA falls within the first range RA1_DIA (40 to 215 mmHg) for the minimum blood pressure DIA, the flag value FDIA for the measured value is 0. Further, since a measured value 61 beats/minute of the pulse rate PLS falls within the first range RA1_PLS (40 to 180 beats/minute) for the pulse rate PLS, the flag value FPLS for the measured value is 0. In this case, a data set in which numerical values of “124”, “0”, “74”, “0”, “61”, and “0” are sequentially arranged is created.

In the example of FIG. 13B, since a measured value 75 mmHg of the maximum blood pressure SYS falls within the first range RA1_SYS (60 to 260 mmHg) for the maximum blood pressure SYS, the flag value FSYS for the measured value is 0. Since a measured value 41 mmHg of the minimum blood pressure DIA falls within the first range RA1_DIA (40 to 215 mmHg) for the minimum blood pressure DIA, the flag value FDIA for the measured value is 0. Further, since a measured value 39 beats/minute of the pulse rate PLS falls within the second range RA2_PLS (30 to 39 beats/minute) for the pulse rate PLS, the flag value FPLS for the measured value is −1. In this case, a data set in which numerical values of “75”, “0”, “41”, “0”, “39”, and “−1” are sequentially arranged is created.

In the example of FIG. 13C, since a measured value 262 mmHg of the maximum blood pressure SYS falls within the fourth range RA4_SYS (261 to 270 mmHg) for the maximum blood pressure SYS, the flag value FSYS for the measured value is 1. Since a measured value 130 mmHg of the minimum blood pressure DIA falls within the first range RA1_DIA (40 to 215 mmHg) for the minimum blood pressure DIA, the flag value FDIA for the measured value is 0. Further, since a measured value 184 beats/minute of the pulse rate PLS falls within the fourth range RA4_PLS (181 to 190 beats/minute) for the pulse rate PLS, the flag value FPLS for the measured value is 1. In this case, a data set in which numerical values of “262”, “1”, “130”, “0”, “184”, and “1” are sequentially arranged is created.

In the example of FIG. 13D, since a measured value 75 mmHg of the maximum blood pressure SYS falls within the first range RA1_SYS (60 to 260 mmHg) for the maximum blood pressure SYS, the flag value FSYS for the measured value is 0. Since a measured value 41 mmHg of the minimum blood pressure DIA falls within the first range RA1_DIA (40 to 215 mmHg) for the minimum blood pressure DIA, the flag value FDIA for the measured value is 0. Further, since a measured value 29 beats/minute of the pulse rate PLS falls within the third range RA3_PLS (29 beats/minute or less) for the pulse rate PLS, the flag value FPLS for the measured value is −2. In this case, a data set in which numerical values of “75”, “0”, “41”, “0”, “29”, and “−2” are sequentially arranged is created.

In the example of FIG. 13E, since a measured value 262 mmHg of the maximum blood pressure SYS falls within the fourth range RA4_SYS (261 to 270 mmHg) for the maximum blood pressure SYS, the flag value FSYS for the measured value is 1. Since a measured value 130 mmHg of the minimum blood pressure DIA falls within the first range RA1_DIA (40 to 215 mmHg) for the minimum blood pressure DIA, the flag value FDIA for the measured value is 0. Further, since a measured value 201 beats/minute of the pulse rate PLS falls within the fifth range RA5_PLS (191 beats/minute or more) for the pulse rate PLS, the flag value FPLS for the measured value is 2. In this case, a data set in which numerical values of “262”, “1”, “130”, “0”, “201”, and “2” are sequentially arranged is created.

In this manner, the control unit 110 creates a data set having a format in which a measured value of the maximum blood pressure SYS, the flag value FSYS for the measured value, a measured value of the minimum blood pressure DIA, the flag value FDIA for the measured value, a measured value of the pulse rate PLS, and the flag value FPLS for the measured value are arranged in this order as information on measured values of a subject (step S114 in FIG. 11).

Subsequently, in step S115 of FIG. 11, the control unit 110 operates the communication unit 90 as a transmission unit to transmit (upload) the created data set to the cloud server 300 illustrated in FIG. 9 via the network 900 in this example in association with an ID (identification number) for identifying the subject. The cloud server 300 stores the data set as the information on measured values in association with the ID (identification number) for identifying the subject.

After the above, when a medical personnel (in this example, a doctor) as the user operates the operation unit 230 of the medical institution terminal 200 to activate application software (computer program) and inputs an instruction to start display processing (including the ID for identifying the subject), the control unit 210 of the medical institution terminal 200 operates the communication unit 290 as a receiving unit and receives the data set as the information on measured values of the subject from the cloud server 300 via the network 900 in step S201 of FIG. 12.

Next, in step S202 of FIG. 12, the control unit 210 acts as a flag recognition unit to read and recognize the measured value of the maximum blood pressure SYS, the flag value FSYS for the measured value, the measured value of the minimum blood pressure DIA, the flag value FDIA for the measured value, the measured value of the pulse rate PLS, and the flag value FPLS for the measured value, which are included in the data set. Then, based on the values of the flag values FSYS, FDIA, and FPLS, the control unit 210 recognizes, for each measurement item, which one of the first, second, . . . , and fifth ranges RA1, RA2, . . . , and RA5 the measured value is classified into.

When the measured value is recognized to be classified into the first range RA1 in step S202, the control unit 210 acts as a first display processing unit to perform processing of displaying the measured value on the display screen (acting as a first display screen in the present embodiment, which similarly applies hereinafter) of the display 250 as illustrated in step S203.

Alternatively, when the measured value is recognized to be classified into the second range RA2 or the fourth range RA4 in step S202, the control unit 210 acts as a first display processing unit to perform processing of displaying, on the display screen of the display 250, deviation information indicating that the measured value is classified into the second range RA2 or the fourth range RA4, or the measured value in addition to the deviation information as illustrated in step S204.

Alternatively, when the measured value is recognized to be classified into the third range RA3 or the fifth range RAS in step S202, the control unit 210 acts as a first display processing unit to display an indication of measurement error with displaying neither the deviation information nor the measured value on the display screen of the display 250 as illustrated in step S205.

In this example, the processing in steps S203 to S205 is performed for each measurement item of the maximum blood pressure SYS, the minimum blood pressure DIA, and the pulse rate PLS (at the same time, the acquired maximum blood pressure SYS, minimum blood pressure DIA, and pulse rate PLS are stored in the memory 220).

For example, in the example of FIG. 14, portion (B), a measured value of the maximum blood pressure SYS is 124 mmHg, which falls within the first range RA1_SYS (60 to 260 mmHg) for the maximum blood pressure SYS. A measured value of the minimum blood pressure DIA is 74 mmHg, which falls within the first range RA1_DIA (40 to 215 mmHg) for the minimum blood pressure DIA. Further, a measured value of the pulse rate PLS is 61 beats/minute, which falls within the first range RA1_PLS (40 to 180 beats/minute) for the pulse rate PLS. In this case, as illustrated in FIG. 14, portion (B), on the display screen of the display 250 of the medical institution terminal 200, the measured value “124” of the maximum blood pressure SYS, the measured value “74” of the minimum blood pressure DIA, and the measured value “61” of the pulse rate PLS are displayed in the right side portion 501b of the maximum blood pressure display region 501, the right side portion 502b of the minimum blood pressure display region 502, and the right side portion 503b of the pulse rate display region 503, respectively. By the above, a doctor can know a measured value for each measurement item. On the other hand, as illustrated in FIG. 14, portion (A), on the display screen of the display 50 of the sphygmomanometer 100 as well, the measured value “124” of the maximum blood pressure SYS, the measured value “74” of the minimum blood pressure DIA, and the measured value “61” of the pulse rate PLS are displayed in the right side portion 501b of the maximum blood pressure display region 501, the right side portion 502b of the minimum blood pressure display region 502, and the right side portion 503b of the pulse rate display region 503, respectively. By the above, a subject can also know a measured value for each measurement item.

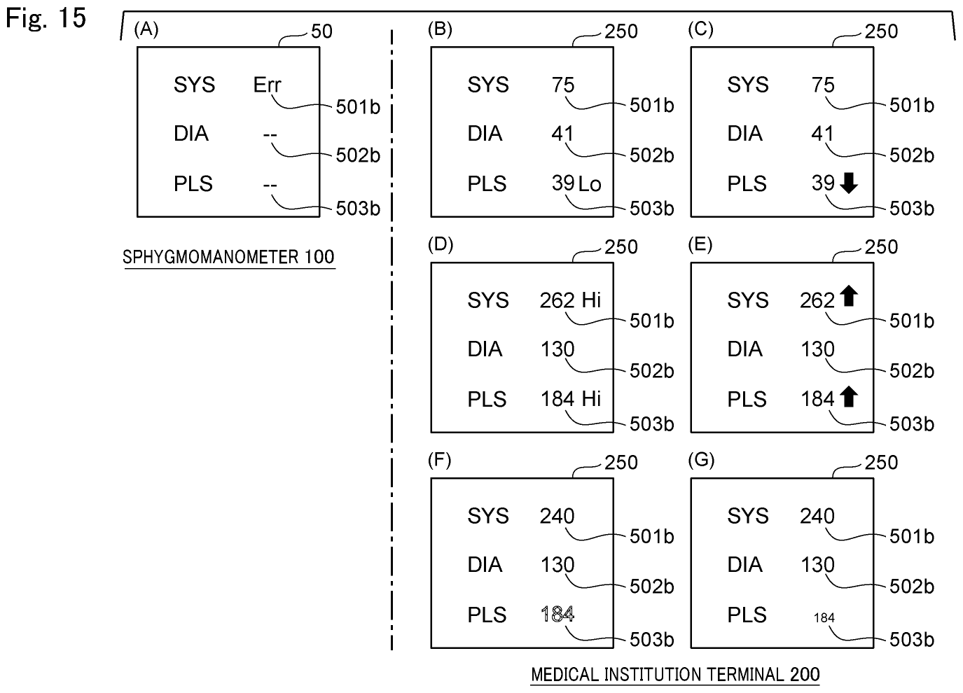

In the examples of FIG. 15, portion (B) and FIG. 15, portion (C), a measured value of the maximum blood pressure SYS is 75 mmHg, which falls within the first range RA1_SYS (60 to 260 mmHg) for the maximum blood pressure SYS. A measured value of the minimum blood pressure DIA is 41 mmHg, which falls within the first range RA1_DIA (40 to 215 mmHg) for the minimum blood pressure DIA. On the other hand, a measured value of the pulse rate PLS is 39 beats/minute, which falls within the second range RA2_PLS (30 to 39 beats/minute) for the pulse rate PLS. In this case, as illustrated in FIG. 15, portion (B) and FIG. 15, portion (C), on the display screen of the display 250 of the medical institution terminal 200, the measured value “75” of the maximum blood pressure SYS and the measured value “41” of the minimum blood pressure DIA are displayed in the right side portion 501b of the maximum blood pressure display region 501 and the right side portion 502b of the minimum blood pressure display region 502, respectively. On the other hand, as illustrated in FIG. 15, portion (B) and FIG. 15, portion (C), for example, the measured value “39” of the pulse rate PLS and deviation information indicating that the measured value of the pulse rate PLS is classified into the second range RA2_PLS is displayed together in the right side portion 503b of the pulse rate display region 503. Here, in the example of FIG. 15, portion (B), the deviation information is represented by the character string “Lo”. In the example of FIG. 15, portion (C), the deviation information is indicated by a downward arrow mark. By the above, a doctor can know the measured value of the maximum blood pressure SYS and the measured value of the minimum blood pressure DIA, and can know that the measured value of the pulse rate PLS deviates to the second range RA2_PLS and the measured value of the pulse rate PLS deviating to the second range RA2_PLS. On the other hand, as illustrated in FIG. 15, portion (A), on the display screen of the display 50 of the sphygmomanometer 100, an indication of measurement error (display of the character string “Err”) is displayed with displaying neither the deviation information nor the measured value. By the above, a subject recognizes that the measured value deviates from the first range RA1 and a measurement error occurs, as in a general electronic sphygmomanometer.

Note that, as illustrated in FIG. 9, a subject can cause communication of the data set to be performed between the sphygmomanometer 100 and the smartphone 400 via the short-range communication line 900N. For example, it is assumed that the display illustrated in FIG. 15, portion (B) (indicated by a balloon DS1 in FIG. 9) is performed on the display screen of the display 250 of the medical institution terminal 200, and the indication illustrated in FIG. 15, portion (A) (indicated by a balloon DS2 in FIG. 9) is displayed on the display screen of the display 50 of the sphygmomanometer 100. In this case, the same indication as that on the display screen of the display 50 of the sphygmomanometer 100 (indicated by a balloon DS3 in FIG. 9) is performed on the display screen of the smartphone 400 by application software (computer program) installed in advance in the smartphone 400. By the above, the subject recognizes that a measured value deviates from the first range RA1 and a measurement error occurs, not only on the display screen of the display 50 of the sphygmomanometer 100 but also on the display screen of the smartphone 400.

In the examples of FIG. 15, portion (D) and FIG. 15, portion (E), a measured value of the maximum blood pressure SYS is 262 mmHg, which falls within the fourth range RA4_SYS (261 to 270 mmHg) for the maximum blood pressure SYS. A measured value of the minimum blood pressure DIA is 130 mmHg, which falls within the first range RA1_DIA (40 to 215 mmHg) for the minimum blood pressure DIA. A measured value of the pulse rate PLS is 184 beats/minute, which falls within the fourth range RA4_PLS (181 to 190 beats/minute) for the pulse rate PLS. In this case, as illustrated in FIG. 15, portion (D), on the display screen of the display 250 of the medical institution terminal 200, for example, a character string “262Hi” obtained by combining the measured value “262” of the maximum blood pressure SYS and the deviation information “Hi” is displayed in the right side portion 501b of the maximum blood pressure display region 501. The measured value “130” of the minimum blood pressure DIA is displayed in the right side portion 502b of the minimum blood pressure display region 502. Further, for example, a character string “184Hi” obtained by combining the measured value “184” for the pulse rate PLS with the deviation information “Hi” is displayed in the right side portion 503b of the pulse rate display region 503. Alternatively, as illustrated in FIG. 15, portion (E), on the display screen of the display 250 of the medical institution terminal 200, for example, the measured value “262” of the maximum blood pressure SYS and an upward arrow mark as deviation information are displayed together in the right side portion 501b of the maximum blood pressure display region 501. The measured value “130” of the minimum blood pressure DIA is displayed in the right side portion 502b of the minimum blood pressure display region 502. Further, for example, the measured value “184” for the pulse rate PLS and the upward arrow mark as deviation information are displayed together in the right side portion 503b of the pulse rate display region 503. By the above, a doctor can know the measured value of the minimum blood pressure DIA, and can also know that the measured value of the maximum blood pressure SYS and the measured value of the pulse rate PLS deviate to the fourth ranges RA4_SYS and RA4_PLS, and the measured value of the maximum blood pressure SYS and the measured value of the pulse rate PLS deviating to the fourth ranges RA4_SYS and RA4_PLS, respectively. On the other hand, as illustrated in FIG. 15, portion (A), on the display screen of the display 50 of the sphygmomanometer 100, an indication of measurement error (display of the character string “Err”) is displayed with displaying neither the deviation information nor the measured value. By the above, a subject recognizes that the measured value deviates from the first range RA1 and a measurement error occurs, as in a general electronic sphygmomanometer.

In the examples of FIG. 15, portion (F) and FIG. 15, portion (G), a measured value of the maximum blood pressure SYS is 240 mmHg, which falls within the first range RA1_SYS (60 to 260 mmHg) for the maximum blood pressure SYS. A measured value of the minimum blood pressure DIA is 130 mmHg, which falls within the first range RA1_DIA (40 to 215 mmHg) for the minimum blood pressure DIA. On the other hand, a measured value of the pulse rate PLS is 184 beats/minute, which falls within the fourth range RA4_PLS (181 to 190 beats/minute) for the pulse rate PLS. In this case, as illustrated in FIG. 15, portion (F) and FIG. 15, portion (G), on the display screen of the display 250 of the medical institution terminal 200, the measured value “240” of the maximum blood pressure SYS and the measured value “130” of the minimum blood pressure DIA are displayed in the first mode in the right side portion 501b of the maximum blood pressure display region 501 and the right side portion 502b of the minimum blood pressure display region 502, respectively. Here, the first mode refers to a predetermined size, thickness, and color in which a measured value is displayed. On the other hand, as illustrated in FIG. 15, portion (F) and FIG. 15, portion (G), a measured value of the pulse rate PLS (“184” in this example) is displayed in the right side portion 503b of the pulse rate display region 503 in the second mode distinguishable from the first mode. In the example of FIG. 15, portion (F), as the second mode, the measured value “184” of the pulse rate PLS is displayed in a color (orange in an actual product, but for convenience of illustration, represented by black hatching on the white background) different from a color (black in this example) of a measured value in the first mode. In the example of FIG. 15, portion (G), as the second mode, the measured value “184” of the pulse rate PLS is displayed with a smaller size than a size at which a measured value is displayed in the first mode and a smaller thickness. By the above, a doctor can know the measured value of the maximum blood pressure SYS and the measured value of the minimum blood pressure DIA, and can know that the measured value of the pulse rate PLS deviates to the fourth range RA4_PLS and the measured value of the pulse rate PLS deviating to the fourth range RA4_PLS. On the other hand, as illustrated in FIG. 15, portion (A), on the display screen of the display 50 of the sphygmomanometer 100, an indication of measurement error (display of the character string “Err”) is displayed with displaying neither the deviation information nor the measured value. By the above, a subject recognizes that the measured value deviates from the first range RA1 and a measurement error occurs, as in a general electronic sphygmomanometer.

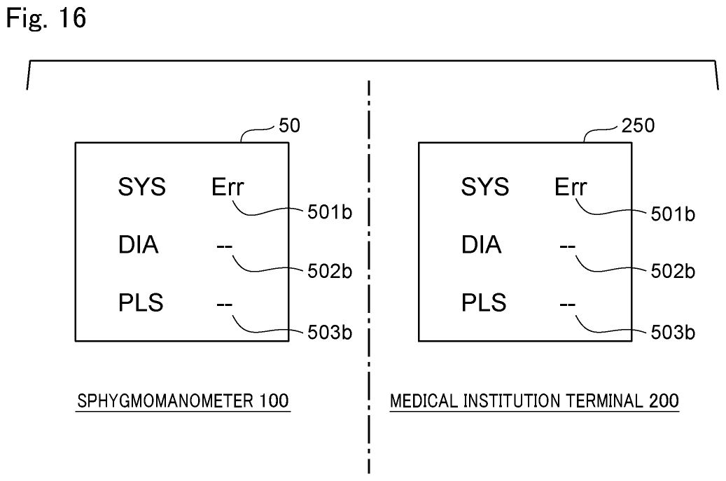

In a case where any one of a measured value of the maximum blood pressure SYS, a measured value of the minimum blood pressure DIA, and a measured value of the pulse rate PLS falls within the third range RA3 or the fifth range RA5, as described in the conventional example, an indication of measurement error (in this example, the character string “Err”) is displayed on the display screen of the display 250 of the medical institution terminal 200 as illustrated in FIG. 16, portion (B). For example, it is assumed that a measured value of the maximum blood pressure SYS is 124 mmHg and falls within the first range RA1_SYS (60 to 260 mmHg) for the maximum blood pressure SYS, and a measured value of the minimum blood pressure DIA is 74 mmHg and falls within the first range RA1_DIA (40 to 215 mmHg) for the minimum blood pressure DIA. Even in this case, for example, when a measured value of the pulse rate PLS is 29 beats/minute and falls within the third range RA3_PLS (29 beats/minute or less) for the pulse rate PLS, not only the measured value of the pulse rate PLS but also the measured value of the maximum blood pressure SYS and the measured value of the minimum blood pressure DIA are not displayed on the display screen of the display 250 of the medical institution terminal 200, and the indication of measurement error as illustrated in FIG. 16, portion (B) is displayed. By the above, a doctor can know that a measurement error occurs. On the other hand, as illustrated in FIG. 16, portion (A), also on the display screen of the display 50 of the sphygmomanometer 100, neither deviation information nor a measured value is displayed, and an indicaton of measurement error (display of the character string “Err”) is displayed with displaying neither the deviation information nor the measured value. By the above, a subject can know that a measurement error occurs. Note that, in the examples of FIG. 16, portion (A) and FIG. 16, portion (B), the character string “Err” is displayed on the right side of the character string “SYS” (the right side portion 501b of the maximum blood pressure display region 501), but the position of the character string “Err” has no meaning as in FIG. 8D.

As described above, according to operation of the medical information management system 800, even in a case where an acquired measured value deviates from the first range RA1 to the lower side (the second range RA2) or the higher side (the fourth range RA4), information on a blood pressure of a subject (deviation information or a measured value in addition to the deviation information) can be displayed on the display screen of the display 250 of the medical institution terminal 200. Therefore, convenience of a medical personnel as the user can be enhanced. In particular, in a case where the deviation information is displayed as a mark (for example, a downward arrow or an upward arrow), a medical personnel can intuitively recognize that a measured value deviates to the second or fourth range RA4 by viewing the mark. Similarly, in a case where the deviation information is displayed in the second mode (for example, a smaller size, a thinner thickness, or a different color) distinguishable from the first mode, a medical personnel can intuitively recognize that a measured value deviates to the second or fourth range RA4 by viewing a measured value displayed in the second mode. Therefore, convenience of a the medical personnel as the user can be further enhanced.

### Variation

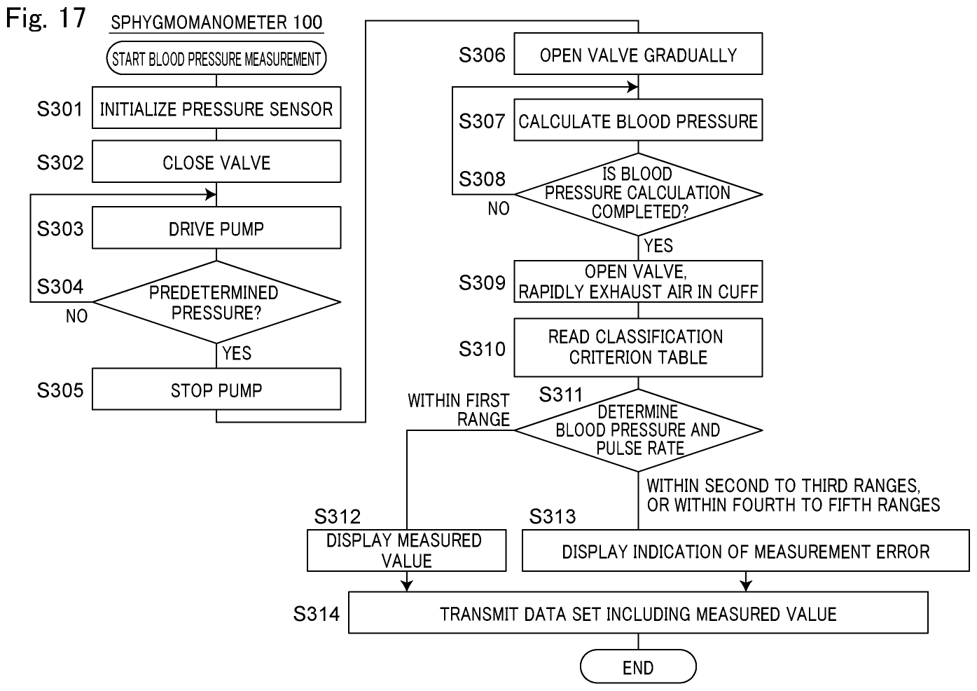

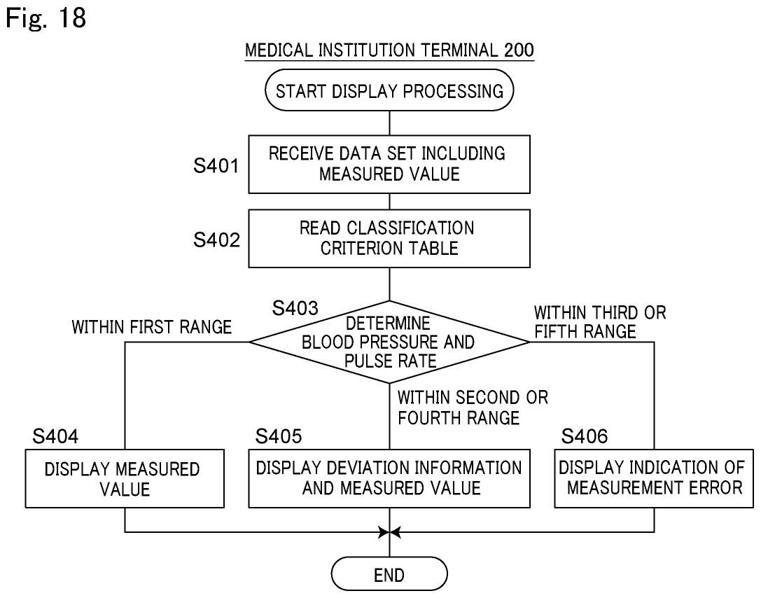

FIG. 17 illustrates another operation flow of the sphygmomanometer 100 included in the medical information management system 800, and FIG. 18 illustrates an operation flow of the medical institution terminal 200 cooperating with the operation flow of FIG. 17 of the sphygmomanometer 100. In this variation, it is assumed that the medical institution terminal 200 stores the same table as the classification criterion table illustrated in FIG. 4A in the memory 220.

When a subject instructs to start measurement with the measurement switch 52A provided on the main body 10 in an attached state where the cuff 20 of the sphygmomanometer 100 is attached to a measurement target site (for example, an upper arm of the subject), the control unit 110 of the sphygmomanometer 100 performs exactly the same processing as steps S1 to S9 of FIG. 2 to acquire a measured value for each of three measurement items of the maximum blood pressure SYS, the minimum blood pressure DIA, and the pulse rate PLS as illustrated in steps S301 to S309 of FIG. 17.

Subsequently, in step S310 of FIG. 17, the control unit 110 reads the classification criterion table of FIG. 4A. Then, in step S311 of FIG. 17, the control unit 110 acts as a second determination unit to refer to the classification criterion table, and to determine whether the acquired measured value is classified into any of the first range RA1, the second to third ranges RA2 to RA3, and the fourth to fifth ranges RA4 to RA5 for each measurement item. Note that, in step S311, the control unit 110 does not need to divide the second range RA2 and the third range RA3, and does not need to divide the fourth range RA4 and the fifth range RA5.

When the measured value is classified into the first range RA1 in step S311, the control unit 110 acts as a second display processing unit to perform processing of displaying the measured value on the display screen (acting as a second display screen in the present variation, which similarly applies hereinafter) of the display 50 as illustrated in step S312.

Alternatively, when the measured value is classified into the second range RA2 to the third range RA3 or the fourth range RA4 to the fifth range RA5 in step S311 of FIG. 17, the control unit 110 acts as a second display processing unit to display an indication of measurement error on the display screen of the display 50 with displaying neither the deviation information nor the measured value as illustrated in step S313.

In this example, the processing in steps S312 to S313 is performed for each measurement item of the maximum blood pressure SYS, the minimum blood pressure DIA, and the pulse rate PLS (at the same time, the acquired maximum blood pressure SYS, minimum blood pressure DIA, and pulse rate PLS are stored in the memory 51). A display example on the display screen of the display 50 will be described later.

By the above, the control unit 110 creates a data set having a format in which a measured value of the maximum blood pressure SYS, a measured value of the minimum blood pressure DIA, and a measured value of the pulse rate PLS are arranged in this order as information on measured values of a subject. Since the flag values FSYS, FDIA, and FPLS are not used, this format is simplified as compared with the format of a data set illustrated in FIG. 13. Therefore, the flag value table of FIG. 4B may be omitted.

Subsequently, in step S314 of FIG. 17, the control unit 110 operates the communication unit 90 as a transmission unit to transmit (upload) the created data set to the cloud server 300 illustrated in FIG. 9 via the network 900 in this example in association with an ID (identification number) for identifying the subject. The cloud server 300 stores the data set as the information on measured values in association with the ID (identification number) for identifying the subject. Note that the subject can upload the data set to the cloud server 300 at a desired timing by turning on the communication switch 52C provided on the main body 10 of the sphygmomanometer 100 even not during an operation flow of blood pressure measurement.

After the above, when a medical personnel (in this example, a doctor) as the user operates the operation unit 230 of the medical institution terminal 200 to activate application software (computer program) and inputs an instruction to start display processing (including the ID for identifying the subject), the control unit 210 of the medical institution terminal 200 operates the communication unit 290 as a receiving unit and receives the data set as the information on measured values of the subject from the cloud server 300 via the network 900 in step S401 of FIG. 18.

Next, in step S402 of FIG. 18, the control unit 210 reads the classification criterion table of FIG. 4A stored in the memory 220. Then, in step S403 of FIG. 18, the control unit 210 acts as a first determination unit to refer to the classification criterion table, and to determine which one of the first, second, . . . , and fifth ranges RA1, RA2, . . . , and RAS the acquired measured value is classified into for each measurement item.

When the measured value is classified into the first range RA1 in step S403, the control unit 210 acts as a first display processing unit to perform processing of displaying the measured value on the display screen (acting as a first display screen in the present variation, which similarly applies hereinafter) of the display 250 as illustrated in step S404.

Alternatively, when the measured value is classified into the second range RA2 or the fourth range RA4 in step S403, the control unit 210 acts as a first display processing unit to perform processing of displaying, on the display screen of the display 250, deviation information indicating that the measured value is classified into the second range RA2 or the fourth range RA4, or the measured value in addition to the deviation information as illustrated in step S405.

Alternatively, when the measured value is classified into the third range RA3 or the fifth range RA5 in step S403, the control unit 210 acts as a first display processing unit to display an indication of measurement error on the display screen of the display 250 with displaying neither the deviation information nor the measured value as illustrated in step S406.

In this example, the processing in steps S404 to S406 is performed for each measurement item of the maximum blood pressure SYS, the minimum blood pressure DIA, and the pulse rate PLS (at the same time, the acquired maximum blood pressure SYS, minimum blood pressure DIA, and pulse rate PLS are stored in the memory 220).

A display example on the display 250 of the medical institution terminal 200 and the display 50 of the sphygmomanometer 100 according to this variation (operation flow of FIGS. 17 and 18) is the same as the display examples described with reference to FIG. 14, portion (A) to FIG. 16, portion (B).

For example, in a case where all measured values of three measurement items of the maximum blood pressure SYS, the minimum blood pressure DIA, and the pulse rate PLS fall within the first range RA1, the display as illustrated in FIG. 14, portion (B) is performed on the display screen of the display 250 of the medical institution terminal 200. By the above, a doctor can know a measured value for each measurement item. On the other hand, the display as illustrated in FIG. 14, portion (A) is also performed on the display screen of the display 50 of the sphygmomanometer 100. By the above, a subject can also know a measured value for each measurement item.

When a measured value of a part of three measurement items of the maximum blood pressure SYS, the minimum blood pressure DIA, and the pulse rate PLS is classified into the first range RAI and a measured value of remaining measurement items is classified into the second range RA2 or the fourth range RA4, the display as illustrated in FIG. 15, portion (B) to FIG. 15, portion (G) is performed on the display screen of the display 250 of the medical institution terminal 200 (note that, when measured values of all measurement items are classified into the second range RA2 or fourth range RA4, the same display is performed for each measurement item). By the above, a doctor can know the measured value of the part of measurement items classified into the first range RAI among measured values of a plurality of measurement items, and can know that the measured value of the remaining measurement items deviates to the second range RA2 or the fourth range RA4 and the measured value deviating to the second range RA2 or the fourth range RA4. On the other hand, the display as illustrated in FIG. 15, portion (A) is performed on the display screen of the display 50 of the sphygmomanometer 100. By the above, a subject recognizes that the measured value deviates from the first range RA1 and a measurement error occurs, as in a general electronic sphygmomanometer.

In a case where any one of a measured value of the maximum blood pressure SYS, a measured value of the minimum blood pressure DIA, and a measured value of the pulse rate PLS falls within the third range RA3 or the fifth range RA5, as described in the conventional example, on the display screen of the display 250 of the medical institution terminal 200, an indication of measurement error (in this example, the character string “Err”) is displayed with displaying neither the deviation information nor the measured value as illustrated in FIG. 16, portion (B). By the above, a doctor can know that a measurement error occurs. On the other hand, as illustrated in FIG. 16, portion (A), also on the display screen of the display 50 of the sphygmomanometer 100, an indication of measurement error (display of the character string “Err”) is displayed with displaying neither the deviation information nor the measured value as illustrated in FIG. 16, portion (A). By the above, a subject can know that a measurement error occurs.

As described above, according to operation (the operation flow of FIGS. 17 and 18) of this variation of the system 800, as in the previous example (the operation flow of FIGS. 11 and 12), even in a case where an acquired measured value deviates from the first range RA1 to the lower side (the second range RA2) or the higher side (the fourth range RA4), information on a blood pressure of a subject (deviation information or a measured value in addition to the deviation information) can be displayed on the display screen of the display 250 of the medical institution terminal 200. Therefore, convenience of a medical personnel as the user can be enhanced. Moreover, in this variation, in the medical institution terminal 200, the control unit 210 acts as a first determination unit to determine which one of the first, second, . . . , and fifth ranges RA1, RA2, . . . , and RA5 the acquired measured value is classified into for each measurement item. Therefore, it is not necessary to include information (for example, a flag value) indicating which range the measured values are classified into as a data set (information on measured values) transmitted by the communication unit 90 of the sphygmomanometer 100. Therefore, processing by the control unit 110 of the sphygmomanometer 100 can be simplified, and communication contents between the communication unit 90 of the sphygmomanometer 100 and the communication unit 290 of the medical institution terminal 200 can be simplified.

In the operation example (the operation flow of FIGS. 11 and 12 or the operation flow of FIGS. 17 and 18) of the system 800 described above, in a case where an acquired measured value deviates from the first range RA1 to the lower side (the second range RA2) or the higher side (the fourth range RA4), information (deviation information or a measured value in addition to the deviation information) on a blood pressure of a subject is displayed on the display screen of the display 250 of the medical institution terminal 200, while on the display screen of the display 50 of the sphygmomanometer 100, an indication of measurement error is displayed with displaying neither the deviation information nor the measured value. However, the present invention is not limited to this. The same contents as the display screen of the display 250 of the medical institution terminal 200 may be displayed on the display screen of the display 50 of the sphygmomanometer 100. This makes it possible to satisfy a subject's need in a case where the subject understands information on a blood pressure.

In the configuration example (see FIG. 9) of the system 800 described above, the sphygmomanometer 100 and the smartphone 400 communicate with the medical institution terminal 200 via the cloud server 300, but the present invention is not limited to this. The sphygmomanometer 100 and the smartphone 400 may directly communicate with the medical institution terminal 200 through the network (in this example, the Internet) 900 without using the cloud server 300. Furthermore, the sphygmomanometer 100 and the smartphone 400 may communicate with the medical institution terminal 200 through a dedicated communication line other than the Internet.

Further, the operation flow of FIG. 12 or the operation flow of FIG. 18 may be recorded as software (computer program) of a blood-pressure-related information display method on a recording medium in which data can be stored in a non-transitory manner such as a compact disc (CD), a digital versatile disc (DVD), or a flash memory. By installing software recorded in such a medium in a substantial computer device such as a personal computer, a personal digital assistant (PDA), or a smartphone, the above-described operation flow can be executed by the computer device.

As described above, a blood-pressure-related information display device of the present disclosure is a blood-pressure-related information display device that displays information related to a blood pressure on a display screen, the blood-pressure-related information display device comprising:
a measured value acquisition unit that acquires a measured value including a systolic blood pressure, a diastolic blood pressure, and/or a pulse rate of a subject;
a determination unit that determines which of three ranges the measured value is classified into, the three ranges being a first range preset in a specification, a second range corresponding to a measured value that can be exhibited by a human, preset adjacent to a side lower than the first range, and a third range corresponding to an abnormal measured value that cannot be exhibited by a human, preset adjacent to a side lower than the second range; and
a display processing unit that performs processing of displaying information on a display screen, wherein
the display processing unit:
displays the measured value on the display screen when the measured value is classified into the first range;
displays deviation information indicating that the measured value is classified into the second range or the measured value in addition to the deviation information on the display screen when the measured value is classified into the second range; and
displays an indication of measurement error on the display screen with displaying neither the deviation information nor the measured value when the measured value is classified into the third range.

A “measured value including a systolic blood pressure, a diastolic blood pressure, and/or a pulse rate” includes a case of a measured value of any one measurement item of a systolic blood pressure, a diastolic blood pressure, and a pulse rate and a case of a measured value of a plurality of measurement items.

To “acquire a measured value” includes, for example, a case of acquiring a measured value by performing measurement in the present device and a case of acquiring a measured value that is measured outside the present device and input via, for example, a communication line.

A “first range set by a specification” may be substantially set by an instruction manual of a device or the like without limitation to a specification sheet.

A “human” refers to a general person, and may be a plurality of persons. A “human” may include a “subject”.

A “second range corresponding to a measured value that may be exhibited by a human, preset adjacent to a side lower than the first range” is preset based on an empirical fact so as to include distribution of measured values that may be exhibited, for example, when a human is sleeping at night, when a human drinks alcohol and a blood vessel expands, when a medicine (for example, a hypotensive agent) taken by a human is too effective, and the like.

Further, a “third range” is preset based on an empirical fact as a range of abnormal measured values that cannot be exhibited by a human.

“Deviation information” may be displayed, for example, as a character, a mark, or the like indicating deviation, or may be displayed as a specific mode of the measured value.

In the blood-pressure-related information display device of the present disclosure, a measured value acquisition unit acquires a measured value including a systolic blood pressure, a diastolic blood pressure, and/or a pulse rate of a subject. A determination unit determines which of three ranges the measured value is classified into, the three ranges being a first range preset in a specification, a second range corresponding to a measured value that can be exhibited by a human, preset adjacent to a side lower than the first range, and a third range corresponding to an abnormal measured value that cannot be exhibited by a human, preset adjacent to a side lower than the second range. A display processing unit displays the measured value on the display screen when the measured value is classified into the first range (original measurement range set in a specification). By the above, the user (including medical personnel such as a doctor and a nurse, and a subject, and this applies similarly hereinafter) can know the measured value. Further, when the measured value is classified into the second range, the display processing unit displays, on the display screen, deviation information indicating that the measured value is classified into the second range, or the measured value in addition to the deviation information. As the deviation information is displayed, the user can know that the measured value deviates to the second range (a range that is lower than an original measurement range and corresponds to a measured value that can be exhibited by a human). Furthermore, as the measured value is displayed in addition to the deviation information, the user can know the measured value deviating to the second range. Further, when the measured value is classified into the third range, the display processing unit displays an indication of measurement error on the display screen with displaying neither the deviation information nor the measured value. By the above, the user can know that a measurement error occurs.

As described above, according to the blood-pressure-related information display device, even in a case where an acquired measured value deviates from the first range to a lower side (second range), information on a blood pressure of a subject (the deviation information, or the measured value in addition to the deviation information) can be displayed. Therefore, convenience of the user can be enhanced.

In the blood-pressure-related information display device according to one embodiment,
the determination unit determines which of five ranges the measured value is classified into, the five ranges being, in addition to the first, second, and third ranges, a fourth range corresponding to a measured value that can be exhibited by a human, preset adjacent to a side higher than the first range, and a fifth range corresponding to an abnormal measured value that cannot be exhibited by a human, preset adjacent to a side higher than the fourth range, and
the display processing unit:
displays deviation information indicating that the measured value is classified into the fourth range or the measured value in addition to the deviation information on the display screen when the measured value is classified into the fourth range, and
displays an indication of measurement error on the display screen with displaying neither the deviation information nor the measured value when the measured value is classified into the fifth range.

A “fourth range corresponding to a measured value that can be exhibited by a human, preset adjacent to a side higher than the first range” is preset based on an empirical fact so as to include distribution of measured values that may be exhibited, for example, when a human is active in the daytime, when a human develops increase in a blood pressure with apnea syndrome during sleep, when a human develops increase in a blood pressure at the time of getting up, and the like.

Further, a “fifth range” is preset based on an empirical fact as a range of abnormal measured values that cannot be exhibited by a human.

In the blood-pressure-related information display device according to this one embodiment, the determination unit determines which of five ranges the measured value is classified into, the five ranges being, in addition to the first, second, and third ranges, a fourth range corresponding to a measured value that can be exhibited by a human, preset adjacent to a side higher than the first range, and a fifth range corresponding to an abnormal measured value that cannot be exhibited by a human, preset adjacent to a side higher than the fourth range. Further, when the measured value is classified into the fourth range, the display processing unit displays, on the display screen, deviation information indicating that the measured value is classified into the fourth range, or the measured value in addition to the deviation information. As the deviation information is displayed, the user can know that the measured value deviates to the fourth range (a range that is higher than an original measurement range and corresponds to a measured value that can be exhibited by a human). Furthermore, as the measured value is displayed in addition to the deviation information, the user can know the measured value deviating to the fourth range. Further, when the measured value is classified into the fifth range, the display processing unit displays an indication of measurement error on the display screen with displaying neither the deviation information nor the measured value. By the above, the user can know that a measurement error occurs.

As described above, according to the blood-pressure-related information display device of this one embodiment, even in a case where an acquired measured value deviates from the first range to a higher side (fourth range), information on a blood pressure of a subject (the deviation information, or the measured value in addition to the deviation information) can be displayed. Therefore, convenience of the user can be enhanced.

In the blood-pressure-related information display device according to one embodiment, the display processing unit:
displays a mark indicating that the measured value is classified into the second or fourth range as the deviation information with respect to the measured value on the display screen when the measured value is classified into the second or fourth range.

A “mark” refers to, for example, an upward or downward arrow.

In the blood-pressure-related information display device according to this one embodiment, when the measured value is classified into the second or fourth range, the display processing unit displays a mark indicating that the measured value is classified into the second or fourth range as the deviation information with respect to the measured value on the display screen. By looking at this mark, the user can intuitively recognize that the measured value deviates to the second or fourth range. Therefore, convenience of the user can be further enhanced.

In the blood-pressure-related information display device according to one embodiment, the display processing unit:
displays the measured value on the display screen in a first mode when the measured value is classified into the first range; and
displays the measured value on the display screen in a second mode distinguishable from the first mode as the deviation information indicating that the measured value is classified into the second or fourth range when the measured value is classified into the second or fourth range.

A “second mode distinguishable from the first mode” refers to, for example, a mode in which size, a color, and the like of a character representing a numerical value are changed from those in the first mode.

In the blood-pressure-related information display device according to this one embodiment, the display processing unit displays the measured value on the display screen in the first mode when the measured value is classified into the first range, and displays the measured value on the display screen in the second mode distinguishable from the first mode as the deviation information indicating that the measured value is classified into the second or fourth range when the measured value is classified into the second or fourth range. By looking at the measured value displayed in the second mode, the user can intuitively recognize that the measured value deviates to the second or fourth range. Furthermore, the user can know the measured value deviating to the second or fourth range. Therefore, convenience of the user can be further enhanced.

In the blood-pressure-related information display device according to one embodiment,
the measured value includes measured values of at least two measurement items of a systolic blood pressure, a diastolic blood pressure, and a pulse rate,
the first to fifth ranges are set in advance for each of the measurement items,
the determination unit determines which of the first to fifth ranges the measured value is classified into for each of the measurement items, and
the display processing unit:
sets a display region for a measurement item for each of the measurement items in the display screen;
displays a measured value in a display region for a certain measurement item in the display screen when the measured value of the certain measurement item is classified into the first range;
displays deviation information indicating that the measured value is classified into the second or fourth range or the measured value in addition to the deviation information in the display region for the certain measurement item when the measured value of the certain measurement item is classified into the second or fourth range; and
displays an indication of measurement error in the display region for the certain measurement item with displaying neither the deviation information nor the measured value when the measured value of the certain measurement item is classified into the third or fifth range.

In the blood-pressure-related information display device according to this one embodiment, the measured value includes measured values of at least two measurement items of a systolic blood pressure, a diastolic blood pressure, and a pulse rate, and the first to fifth ranges are set in advance for each of the measurement items. In accordance with the above, the determination unit determines which one of the first to fifth ranges the measured value is classified into for each of the measurement items. The display processing unit sets a display region for a measurement item for each of the measurement items in the display screen, and performs the above-described displaying processing for each display region of each measurement item. Therefore, convenience of the user can be further enhanced.

In the blood-pressure-related information display device according to one embodiment,
when, among the measured values, a measured value of a part of measurement items is classified into the first range and a measured value of remaining measurement items is classified into the second or fourth range by the determination unit,
the display processing unit:
displays the measured value of the part of measurement items classified into the first range in a display region for the part of measurement items in the display screen; and
displays only the deviation information without displaying the measured value of the remaining measurement items classified into the second or fourth range in a display region for the remaining measurement items in the display screen.

In the blood-pressure-related information display device according to this one embodiment, when, among the measured values, a measured value of a part of measurement items is classified into the first range and a measured value of remaining measurement items is classified into the second or fourth range by the determination unit, the display processing unit displays the measured value of the part of measurement items classified into the first range in a display region for the part of the measurement items in the display screen. Together with the above, the display processing unit displays only the deviation information without displaying the measured value of the remaining measurement items classified into the second or fourth range in a display region for the remaining measurement items in the display screen. Therefore, the user can know the measured value of the part of measurement items classified in the first range among measured values of a plurality of measurement items, and can know that the measured value of the remaining measurement items deviates to the second or fourth range.

The blood-pressure-related information display device according to one embodiment further comprises:
a sphygmomanometer main body, wherein
a display forming the display screen, the measured value acquisition unit, the determination unit, and the display processing unit are mounted on the sphygmomanometer main body.

In the blood-pressure-related information display device of this one embodiment, in the sphygmomanometer main body, processing of determination by the determination unit and display processing by the display processing unit are performed on the measured value acquired by the measured value acquisition unit. When the measured value is classified into the first range, the measured value is displayed on the display screen of the display mounted on the sphygmomanometer main body. By the above, a subject as the user can know the measured value. Further, when the measured value is classified into the second or fourth range, deviation information indicating that the measured value is classified into the second or fourth range, or the measured value in addition to the deviation information is displayed on the display screen. As the deviation information is displayed, a subject can know that the measured value deviates to the second or fourth range. Furthermore, as the measured value is displayed in addition to the deviation information, a subject can know the measured value deviating to the second or fourth range. Further, when the measured value is classified into the third or fifth range, neither the deviation information nor the measured value is displayed on the display screen, and an indication of measurement error is displayed with displaying neither the deviation information nor the measured value. By the above, a subject can know that a measurement error occurs. Therefore, convenience of a subject as the user can be enhanced.

The blood-pressure-related information display device according to one embodiment further comprises:
a sphygmomanometer; and
a medical institution terminal provided in a manner separated from the sphygmomanometer, wherein
the sphygmomanometer includes:
the measured value acquisition unit that measures and acquires the measured value including the systolic blood pressure, the diastolic blood pressure, and/or the pulse rate from the subject; and
a transmission unit that transmits information on the measured value acquired by the measured value acquisition unit, and
the medical institution terminal includes:
a receiving unit that receives the information on the measured value from the sphygmomanometer;
a display that forms a first display screen acting as the display screen; and
a first display processing unit that acts as the display processing unit that performs processing of displaying information on the first display screen based on the information on the measured value.

A “medical institution terminal” refers to a terminal (substantial computer device such as a personal computer, a tablet, or a smartphone) managed and used by a medical personnel (doctor, nurse, or the like) engaged in a medical institution (hospital, clinic, or the like), not by a subject.

“Information on the measured value” may be, for example, information itself indicating the measured value, or may include information indicating which range the measured value is classified into, in addition to the information indicating the measured value.

A blood-pressure-related information display device according to this one embodiment includes a sphygmomanometer and a medical institution terminal provided in a manner separated from the sphygmomanometer. In the sphygmomanometer, the measured value acquisition unit measures and acquires the measured value including the systolic blood pressure, the diastolic blood pressure, and/or the pulse rate from the subject. The transmission unit transmits information on the measured value acquired by the measured value acquisition unit to the medical institution terminal. In the medical institution terminal, the receiving unit receives the information on the measured value from the sphygmomanometer. A first display processing unit acting as the display processing unit performs processing of displaying information on the first display screen as the display screen on the basis of the information on the measured value. Therefore, the first display processing unit can perform the above-described display processing on the first display screen. By the above, even in a case where an acquired measured value deviates from the first range to the lower side (second range), or in addition, deviates from the first range to the higher side (fourth range), the user, particularly a medical personnel engaged in the medical institution, can know the information on the blood pressure of the subject (the deviation information or the measured value in addition to the deviation information). Therefore, convenience of a medical personnel as the user can be enhanced.

Note that, in the blood-pressure-related information display device of this one embodiment, spatial arrangement of the “determination unit” is not limited. Therefore, the “determination unit” may be mounted on, for example, the sphygmomanometer, the medical institution terminal, or a server connected to the sphygmomanometer and the medical institution terminal via a communication line.

In the blood-pressure-related information display device according to one embodiment,
the medical institution terminal further includes a first determination unit that acts as the determination unit.

In the blood-pressure-related information display device of this one embodiment, in the medical institution terminal, the first determination unit acting as the determination unit determines which of five ranges from the first to fifth ranges the measured value is classified into, based on the information on the measured value. Therefore, it is not necessary to include information indicating which range the measured value is classified into as the information on the measured value transmitted by the transmission unit of the sphygmomanometer. Therefore, processing in the sphygmomanometer can be simplified, and communication contents between the transmission unit of the sphygmomanometer and the receiving unit of the medical institution terminal can be simplified.

In the blood-pressure-related information display device according to one embodiment,
the sphygmomanometer further includes a second determination unit, a display forming a second display screen, and a second display processing unit that performs processing of displaying information on the second display screen,
the second determination unit determines whether the measured value is classified into any of the first range, the second to third ranges, and the fourth to fifth ranges, and
the second display processing unit:
displays the measured value on the second display screen when the measured value is classified into the first range; and
displays an indication of measurement error on the second display screen with displaying neither the deviation information nor the measured value when the measured value is classified into the second to third ranges or the fourth to fifth ranges.

In the blood-pressure-related information display device according to this one embodiment, in the sphygmomanometer, the second determination unit determines whether the measured value is classified into any of the first range, the second to third ranges, and the fourth to fifth ranges. The second display processing unit displays the measured value on the second display screen when the measured value is classified into the first range. A subject as the user can know the measured value. Further, when the measured value is classified into the second to third ranges or the fourth to fifth ranges, the second display processing unit displays an indication of measurement error on the second display screen with displaying neither the deviation information nor the measured value. By the above, a subject recognizes that the measured value deviates from the first range and a measurement error occurs, as in a general electronic sphygmomanometer.

As is clear from the above, according to the blood-pressure-related information display device of the present disclosure, information on a blood pressure of a subject can be displayed even in a case where an acquired measured value deviates from a measurement range (first range) set in advance in a specification.

The above embodiments are illustrative, and are modifiable in a variety of ways without departing from the scope of this invention. It is to be noted that the various embodiments described above can be appreciated individually within each embodiment, but the embodiments can be combined together. It is also to be noted that the various features in different embodiments can be appreciated individually by its own, but the features in different embodiments can be combined.

1. A blood-pressure-related information display device that displays information related to a blood pressure on a display screen, the blood-pressure-related information display device comprising:
a measured value acquisition unit that acquires a measured value including a systolic blood pressure, a diastolic blood pressure, and/or a pulse rate of a subject;
a determination unit that determines which of three ranges the measured value is classified into, the three ranges being a first range preset in a specification, a second range corresponding to a measured value that can be exhibited by a human, preset adjacent to a side lower than the first range, and a third range corresponding to an abnormal measured value that cannot be exhibited by a human, preset adjacent to a side lower than the second range; and
a display processing unit that performs processing of displaying information on a display screen, wherein
the display processing unit:
displays the measured value on the display screen when the measured value is classified into the first range;
displays deviation information indicating that the measured value is classified into the second range or the measured value in addition to the deviation information on the display screen when the measured value is classified into the second range; and
displays an indication of measurement error on the display screen with displaying neither the deviation information nor the measured value when the measured value is classified into the third range.

2. The blood-pressure-related information display device according to claim 1, wherein
the determination unit determines which of five ranges the measured value is classified into, the five ranges being, in addition to the first, second, and third ranges, a fourth range corresponding to a measured value that can be exhibited by a human, preset adjacent to a side higher than the first range, and a fifth range corresponding to an abnormal measured value that cannot be exhibited by a human, preset adjacent to a side higher than the fourth range, and
the display processing unit:
displays deviation information indicating that the measured value is classified into the fourth range or the measured value in addition to the deviation information on the display screen when the measured value is classified into the fourth range, and
displays an indication of measurement error on the display screen with displaying neither the deviation information nor the measured value when the measured value is classified into the fifth range.

3. The blood-pressure-related information display device according to claim 2, wherein
the display processing unit:
displays a mark indicating that the measured value is classified into the second or fourth range as the deviation information with respect to the measured value on the display screen when the measured value is classified into the second or fourth range.

4. The blood-pressure-related information display device according to claim 2, wherein
the display processing unit:
displays the measured value on the display screen in a first mode when the measured value is classified into the first range; and
displays the measured value on the display screen in a second mode distinguishable from the first mode as the deviation information indicating that the measured value is classified into the second or fourth range when the measured value is classified into the second or fourth range.

5. The blood-pressure-related information display device according to claim 2, wherein
the measured value includes measured values of at least two measurement items of a systolic blood pressure, a diastolic blood pressure, and a pulse rate,
the first to fifth ranges are set in advance for each of the measurement items,
the determination unit determines which of the first to fifth ranges the measured value is classified into for each of the measurement items, and
the display processing unit:
sets a display region for a measurement item for each of the measurement items in the display screen;
displays a measured value in a display region for a certain measurement item in the display screen when the measured value of the certain measurement item is classified into the first range;
displays deviation information indicating that the measured value is classified into the second or fourth range or the measured value in addition to the deviation information in the display region for the certain measurement item when the measured value of the certain measurement item is classified into the second or fourth range; and
displays an indication of measurement error in the display region for the certain measurement item with displaying neither the deviation information nor the measured value when the measured value of the certain measurement item is classified into the third or fifth range.

6. The blood-pressure-related information display device according to claim 5, wherein
when, among the measured values, a measured value of a part of measurement items is classified into the first range and a measured value of remaining measurement items is classified into the second or fourth range by the determination unit,
the display processing unit:
displays the measured value of the part of measurement items classified into the first range in a display region for the part of measurement items in the display screen; and
displays only the deviation information without displaying the measured value of the remaining measurement items classified into the second or fourth range in a display region for the remaining measurement items in the display screen.

7. The blood-pressure-related information display device according to claim 2, further comprising:
a sphygmomanometer main body, wherein
a display forming the display screen, the measured value acquisition unit, the determination unit, and the display processing unit are mounted on the sphygmomanometer main body.

8. The blood-pressure-related information display device according to claim 2, further comprising:
a sphygmomanometer; and
a medical institution terminal provided in a manner separated from the sphygmomanometer, wherein
the sphygmomanometer includes:
the measured value acquisition unit that measures and acquires the measured value including the systolic blood pressure, the diastolic blood pressure, and/or the pulse rate from the subject; and
a transmission unit that transmits information on the measured value acquired by the measured value acquisition unit, and
the medical institution terminal includes:
a receiving unit that receives the information on the measured value from the sphygmomanometer;
a display that forms a first display screen acting as the display screen; and
a first display processing unit that acts as the display processing unit that performs processing of displaying information on the first display screen based on the information on the measured value.

9. The blood-pressure-related information display device according to claim 8, wherein
the medical institution terminal further includes a first determination unit that acts as the determination unit.

10. The blood-pressure-related information display device according to claim 8, wherein
the sphygmomanometer further includes a second determination unit, a display forming a second display screen, and a second display processing unit that performs processing of displaying information on the second display screen,
the second determination unit determines whether the measured value is classified into any of the first range, the second to third ranges, and the fourth to fifth ranges, and
the second display processing unit:
displays the measured value on the second display screen when the measured value is classified into the first range; and
displays an indication of measurement error on the second display screen with displaying neither the deviation information nor the measured value when the measured value is classified into the second to third ranges or the fourth to fifth ranges.
